(19) United States
(12) Patent Application Publication
(10) Pub. No.: US 2007/0082041 A1
(43) Pub. Date: Apr. 12, 2007

(54) Topical delivery of codrugs

(75) Inventors: Kenneth A. Walters, Loughborough (GB); Robert Shimizu, Acton, MA (US); Paul Ashton, Boston, MA (US); Grazyna Cynkowska, Brookline, MA (US); Tadeusz Cynkowski, Brookline, MA (US); Eric Gauthier, Waltham, MA (US)

Correspondence Address: FISH & NEAVE IP GROUP;ROPES & GRAY LLP, ONE INTERNATIONAL PLACE, BOSTON, MA, 02110-2624, US

(73) Assignee: pSivida Inc., Watertown, MA (US)

(21) Appl. No.: 11/636,774

(22) Filed: Dec. 11, 2006

Related U.S. Application Data
(63) Continuation of application No. 10/299,597, filed on Nov. 19, 2002, now abandoned.
(60) Provisional application No. 60/331,512, filed on Nov. 19, 2001.

(51) Int. Cl. A61K 31/58; A61K 38/19; A61K 9/70

(52) U.S. Cl. 424/449; 514/171; 424/85.1

(57) ABSTRACT

The present invention provides pharmaceutical compositions for topical delivery comprising a suitable carrier and a codrug capable of penetrating, or being transported across, the dermis. The codrug according to the invention comprises a first constituent moiety linked to a second constituent moiety, wherein the second constituent moiety is the same as, or different from, the first constituent moiety. The first and second constituent moieties are so linked that they are easily transported into or across the dermis, into the skin, or into the blood or lymphatic system, and are reconstituted in vivo to form the first and second constituent moieties.

### CROSS-REFERENCE TO RELATED APPLICATIONS

This application claims the benefit of priority from U.S. Provisional Application No. 60/331,512, filed Nov. 19, 2001, the specification of which is incorporated by reference herein in its entirety.

### FIELD OF THE INVENTION

The present invention relates to the field of topical delivery of pharmaceutical compositions. In particular, the present invention relates to topical delivery of codrugs.

### BACKGROUND OF THE INVENTION

Methods of delivering biologically active moieties to a patient include intravenous, subcutaneous, intraperitoneal, epidural, intramuscular, oral, and topical administration. Each mode of administration comes with its own set of drawbacks. For instance, parenteral administration, such as intravenous administration, requires the attention of a medical professional, such as a registered nurse. All modes of parenteral administration require the use of needles and syringes, which is generally undesirable, except when the patient is bedfast or unable to swallow medication. While oral administration is attractive, it requires that the pharmaceutical composition be stable in the gut and have good uptake characteristics from the intestines. Generally the latter requirement means that the pharmaceutical composition must be somewhat water soluble within the pH range of the small intestine.

Topical administration of biologically active moieties is becoming increasingly popular as an area of investigative interest. One known method is the combining of certain biologically active moieties with one or more carriers and the application of the combination to the skin for local or systemic treatment of certain conditions. For instance, hydrocortisone has long been used in a cream or ointment base for topical application to the skin for local treatment of inflammation, itching, and/or pain associated with localized skin irritation. Antiemetics, such as scopolamine, have been applied transdermally via a patch for the prevention or treatment of nausea. However, the topical mode of drug administration is limited by the ability of potential drug candidates to be absorbed by, or cross, the dermal barrier.

Nevertheless, it would be helpful to be able to administer a wide variety of biologically active moieties via a topical route, either in the form of a lotion, cream or other semi-solid vehicle, or via a sustained-release topical medical device, such as a slow-release patch. Heretofore, treatment of a number of medical disorders via topical application of therapeutic compositions has been difficult because of poor penetration of the compositions into the skin.

There is a need for improved moieties and compositions for the dermal or transdermal delivery of biologically active moieties, such as antiproliferative, antipsoriatic, antiinflammatory, anxiolytic, antidepressant, antipsychotic, antibiotic, and anti-pain moieties to a patient in need of treatment with such moieties.

### SUMMARY OF THE INVENTION

The present invention provides a pharmaceutical composition for local dermal or transdermal systemic delivery of at least one biologically active moiety, the composition comprising: a pharmaceutically acceptable carrier; and a codrug capable of being absorbed by, or transported across, the epidermal layer, wherein the codrug is relatively lipophilic and the constituent moieties are reconstituted after the codrug has been absorbed by, or transported across, the epidermal layer.

Embodiments of the present invention further provide a method of delivering one or more biologically active moieties to a patient in need thereof, the method comprising: applying to the skin of a patient in need thereof a pharmaceutically effective amount of a pharmaceutical composition, the composition comprising: a pharmaceutically acceptable carrier; and a codrug capable of being absorbed by, or transported across, the epidermal layer, wherein the codrug is relatively lipophilic and the constituent moieties are reconstituted after the codrug has been absorbed by, or transported across, the epidermal layer.

Embodiments according to the present invention provide a device for delivery of at least one biologically active moiety into or through the skin, the device comprising a composition according to the present invention and a means for releasing the codrug into or across the skin.

One aspect of the present invention provides a codrug having excellent skin penetration and/or permeability characteristics, the codrug being capable of penetrating the skin and delivering to a patient in need thereof one or more constituent moieties for the treatment of a medical disorder.

Another aspect of the present invention provides a pharmaceutical composition for topical application to a patient in need of medicinal treatment, the composition comprising one or more vehicles in admixture with one or more of said codrugs.

Yet another aspect of the present invention provides medical devices capable of delivering to a patient in need of such treatment one or more of said codrugs.

A further aspect of the present invention provides therapeutic methods of treatment, the methods comprising administering to a person in need of such treatment one or more of said codrugs.

The pharmaceutical compositions according to the present invention offer the advantage of improved skin penetration and/or permeability, and hence improved bioavailability, via the topical administration route, as compared to at least one constituent moiety. The pharmaceutical compositions according to the present invention also offer the advantage of possessing more favorable skin residence characteristics as compared to at least one of the constituent moieties.

Other aspects and advantages of the present invention will become apparent to the person having skill in the art upon consideration of the following description, claims, and abstract of the disclosure.

### DETAILED DESCRIPTION OF THE INVENTION

I. Overview

The present invention is provides pharmaceutical compositions for dermal (local) and transdermal (systemic) delivery of codrugs. The present invention addresses shortcomings in the art by delivering one or more constituent moieties either locally or systemically via a codrug intermediate that passes into or through the skin. Each molecule of the codrug comprises at least two, and as many as three, four, or five, molecules of constituent moieties. The codrug has the property that it is more lipophilic than the constituent moieties, and thus is able to penetrate and/or traverse the skin (epidermis) better than the constituent moieties. The codrug has the further property that, once the codrug has been exposed to in vivo aqueous environments, either within cells or in various aqueous biological media, such as blood, interstitial fluid, lymphatic fluid, etc., the codrug is hydrolyzed to form the constituent moieties. The present invention thus contemplates effective transport of the constituent moieties as part of the codrug, and effective delivery of biologically active constituent moieties in vivo.

The compositions according to the present invention are especially useful for delivering pharmaceutically active moieties either directly to the skin, or transdermally for systemic delivery of the pharmaceutically active moieties.

One aspect of the present invention provides a pharmaceutical composition comprising a codrug, a pharmaceutically acceptable salt, or prodrug thereof, for topical administration of at least one biologically active moiety, which codrug comprises:
a) at least two constituent moieties, each moiety being a residue of a biologically active compound or a prodrug thereof, including a first constituent moiety and a second constituent moiety;
b) a linkage covalently linking said at least two constituent moieties to form said codrug, said linkage is cleaved under physiological conditions after the codrug has been transported into or across the epidermal layer to regenerate said constituent moieties;

wherein the pH of the composition is less than about 7 and the codrug exhibits improved dermal uptake relative to at least one of the constituent moieties.

Another aspect of the invention provides a pharmaceutical composition comprising a codrug, a pharmaceutically acceptable salt, or prodrug thereof, for topical administration of at least one biologically active moiety, which codrug comprises:
a) at least two constituent moieties, each moiety being a residue of a biologically active compound or a prodrug thereof, including a first constituent moiety and a second constituent moiety;
b) a linkage covalently linking said at least two constituent moieties to form said codrug, said linkage is cleaved under physiological conditions after the codrug has been transported into or across the epidermal layer to regenerate said constituent moieties;

wherein the codrug has a log P value from about 1 to about 8, and the codrug exhibits improved dermal uptake relative to at least one of the constituent moieties.

In certain embodiments, at least one of the constituent moieties has a log P value at least 1 log P unit less than the log P value of the codrug. In some embodiments, at least one of the constituent moieties has a log P value at least 1.5 log P units, and preferable at least 2 log P units, less than the log P value of the codrug.

In certain embodiments of the invention, the codrug has a log P value from about 1 to about 3. In other embodiments of the invention, the codrug has a log P value from about 3 to about 6.

In some embodiments, first constituent moiety is selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds.

In certain embodiments, the second constituent moiety is selected from antidepressant compounds, analgesic compounds, antiinflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds.

Yet another aspect of the invention provides a pharmaceutical composition according to claim 1 or 2, wherein the codrug has the following structural formula:
R₁−L−(R₂)ₙ

wherein the first constituent moiety is R₁;

the second constituent moiety is R₂;

R₁ and R₂ each represent, independently, a residue of a compound selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds;

n is an integer of from 1 to 4;

and L is selected from a direct bond and a linking group;

wherein the codrug has a log P value from about 1 to about 8, and the codrug exhibits improved dermal uptake relative to at least one of the constituent moieties.

Still yet another aspect of the invention provides a pharmaceutical composition according to claim 1 or 2, wherein the codrug has the following structural formula:
R₁−(L−R₂)ₙ

wherein the first constituent moiety is R₁;

the second constituent moiety is R₂;

R₁ and R₂ each represent, independently, a residue of a compound selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds;

n is an integer of from 1 to 4;

and L is selected from a direct bond and a linking group;

wherein the codrug has a log P value from about 1 to about 8, and the codrug exhibits improved dermal uptake relative to at least one of the constituent moieties.

Still yet another aspect of the invention provides a pharmaceutical composition according to claim 1 or 2, wherein the codrug has the following structural formula:
(R₁−L)ₘR₂(L₂−R₃)ₙ

wherein R₁, R₂, L, m, and n are defined as above;

R₃ represents a residue of a compound selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds;

and L₂ may be a linking group the same as or different from L. It should be noted that the counterion or salt may, itself, have pharmacological activity.

Preferred values of n and m above are 1 or 2.

Preferably, R₁ and R₂ each represent, independently, a residue of a compound selected from analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, skin-treating compounds, sunscreens, skin protectants, antimetabolite compounds, antipsoriatic compounds, and keratolytic compounds. More preferably, R₁ and R₂ each represent, independently, a residue of a compound selected from anti-inflammatory steroidal compounds (corticosteroids), non-steroidal antiinflammatory compounds (NSAIDs), anti-fungal compounds, antiproliferative compounds, skin-treating compounds, sunscreens, skin protectants, antimetabolite compounds, antipsoriatic compounds, and keratolytic compounds. Even more preferably, R₁ and R₂ each represent, independently, a residue of a compound selected from anti-inflammatory steroidal compounds (corticosteroids), non-steroidal antiinflammatory compounds (NSAIDs), antiproliferative compounds, antimetabolite compounds, antipsoriatic compounds, and keratolytic compounds.

In certain embodiments, R₂ is a residue of diclofenac, etodolac, ketorolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or salts thereof.

In some embodiments, R₁ is a residue of alitretinoin (9-cis-retinoic acid); amifostine; bexarotene(4-[1-(5,6,7,8-tetrahydro-3,5,5,8,8-pentamethyl-2-naphthalenyl)ethenyl] benzoic acid); bleomycin; capecitabine(5′-deoxy-5-fluoro-cytidine); chlorambucil; bleomycin; BCNU; cladribine; cytarabine; daunorubicin; docetaxel; doxorubicin; epirubicin; estramustine; etoposide; exemestane(6-methylenandrosta-1,4-diene-3,17-dione); fludarabine; 5-fluorouracil; gemcitabine; hydroxyurea; idarubicin; irinotecan; melphalan; methotrexate; mitoxantrone; paclitaxel; pentostatin; streptozocin; temozolamide; teniposide; tomudex; topotecan; valrubicin(N-trifluoroacetyladriamycin-14-valerate); or vinorelbine.

In some embodiments, R₂ is a residue of:

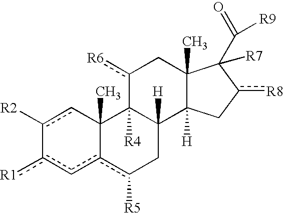

wherein R1 is ═O, —OH, or —(CH₂)₁₋₄Cl;

R2 is H, C₁₋₄alkyl, Cl, or Br;

R4 is H, F, or Cl;

R5 is H, F, Cl, CH₃, or —CHO;

R6 is H, OH, or Cl;

R7 is H, OH, CH₃, O—COCH₃, O(CO)OCH₂CH₃, O—(CO)-2-furanyl, or O—C(O)—(CH₂)₂CH₃;

R8 is H, CH₃, OH, ═CH₂, or together R7 and R8 form, together with the adjacent carbon atoms to which they are attached:

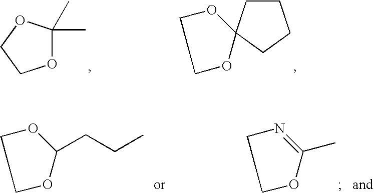

R9 is CH₃, CH₂OH, CH₂O(CO)CH₃, CH₂—O—Cl₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)-NH(CO)C₆H₅, or —S—CH₂—F; and

wherein the bonds indicated by

[symbol]

are either double or single bonds.

In certain embodiments, R₂ is a residue of 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts thereof.

In some embodiments, the pharmaceutical composition according to claim 1 or 2, further comprises a carrier, an excipient, a solvent, an adjuvant, a diluent, a dispersant, or a surfactant. In certain embodiments, the carrier comprises a biocompatible polymer. In some embodiments, the polymer comprises PVA.

In certain embodiments, the composition has a pH of less than about 6. In some embodiments, the composition has a pH from about 1 and about 7, preferably from about 2 to about 6, and more preferably from about 4 to about 6.

In some embodiments, the composition is for local dermal delivery. In other embodiments, the composition is for systemic transdermal delivery.

In some embodiments, the first constituent moiety is the same as the second constituent moiety. In other embodiments, the first constituent moiety is different from the second constituent moiety.

In some embodiments, the first constituent moiety has a similar potency as the second constituent moiety. In certain embodiments, the potency of each of the constituent moieties is measured by EC₅₀. In certain preferred embodiments, the ratio of EC₅₀ of the first constituent moiety to the EC₅₀ of the second constituent moiety is about 1. In other preferred embodiments, the ratio of EC₅₀ of the first constituent moiety to the EC₅₀ of the second constituent moiety is from about 1 to about 4.

In some embodiments, the first and second constituent moieties are directly linked through a covalent bond formed between a functional group of the first constituent moiety and a functional group of the second constituent moiety. In other embodiments, the first and second constituent moieties are linked to one another via a linking group that is covalently bonded to the first and second constituent moieties via functional groups thereon.

In certain embodiments, the first constituent moiety is a corticosteroid. In some embodiments, the second constituent moiety is a corticosteroid, a keratolytic compound, a skin-treating compound, an antiproliferative compound, or a non-steroidal anti-inflammatory compound.

In some embodiments, the corticosteroid is selected from triamcinolone acetonide, fluocinolone acetate, fluocinolone acetonide, cortisone, hydrocortisone, and hydrocortisone ester.

In some embodiments, wherein the first constituent moiety is an antiproliferative agent and the second constituent moiety is a non-steroidal anti-inflammatory agent. In certain such embodiments, the first constituent moiety is not floxuridine, and with the further proviso that when the first constituent moiety is 5-fluororuracil, the second constituent moiety is not flurbiprofen or indomethacin.

In certain embodiments, the first constituent moiety is an antiproliferative agent and the constituent moiety is a corticosteroid agent. In certain such embodiments, when the antiproliferative agent is 5-fluorouracil, the corticosteroid is not fluocinolone acetonide, triamcinolone, triamcinolone acetonide, desoximetasone, or hydrocortisone-17-butyrate, and with the further proviso that the antiproliferative agent is not a 1-β-arabinofuranosylcytosine derivative.

Another aspect of the invention provides a method of treatment, comprising administering to a patient in need thereof a therapeutically effective amount of a composition according to claim 1 or 2, or a pharmaceutically acceptable salt thereof.

In some embodiments, the therapeutically effective amount is an amount effective to produce an analgesic, an anti-inflammatory, an antibiotic, an anti-fungal, an antiviral, an antiproliferative, a skin-treating, a sunscreen, a skin protecting, an antimetabolite, an antipsoriatic, and/or keratolytic effect. More preferably, the therapeutically effective amount is an amount effective to produce an analgesic, an anti-inflammatory, an antiproliferative, a sunscreen, a skin protecting, an antimetabolite, an antipsoriatic, and/or keratolytic effect. In certain embodiments, the therapeutically effective amount is an amount effective to produce an anti-inflammatory, an antiproliferative, a skin-treating, a sunscreen, a skin protecting, an antimetabolite, an antipsoriatic, and/or keratolytic effect.

Yet still another aspect of the invention provides a device for delivery of one or more biologically active moieties to or through the skin, the device comprising a composition according to claim 1 and a means for releasing the codrug into or across the skin.

In certain embodiments, the means for releasing the codrug into or across the skin is a microneedle, a bandage, a gauze pad, or a patch.

In some embodiments, the means for releasing the codrug into or across the skin is a patch, said patch comprising an impermeable backing layer, a permeable skin contact layer, and a reservoir containing said composition. In certain embodiments, the reservoir comprises one or more solvents, permeability enhancers, hydrogels, or non-hydrophilic polymers.

The linker L may be either a direct bond between individual constituent moieties, or it may include a linking group. The first and second constituent moieties of the codrugs of the present invention may be linked via reversible covalent bonds such as ester, amide, carbamate, carbonate, cyclic ketal, thioester, thioamide, thiocarbamate, thiocarbonate, xanthate and phosphate ester bonds, so that, at the required site in the body, they are cleaved to regenerate the active forms of the constituent pharmaceutically active agents.

The covalent bonds between residues include a bonding structure such as:

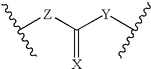

wherein Z is O, N, —CH₂—, —CH₂—O— or —CH₂—S—, Y is O, or N, and X is O or S. The rate of cleavage of the individual moieties can be controlled by the type of bond, the choice of constituent moieties, and the physical form of the codrug. The lability of the selected bond type may be enzyme-specific. In some embodiments according to the present invention, the bond is selectively labile in the presence of an esterase. In other embodiments of the invention, the bond is chemically labile, e.g., to acid- or base-catalyzed hydrolysis.

In preferred embodiments according to the present invention, the linking group L does not include a sugar, a reduced sugar, a pyrophosphate, or a phosphate group.

The physiologically labile linkage may be any linkage that is labile under conditions approximating those found in physiologic fluids, such as is found in the dermis or viable epidermis. The linkage may be a direct bond (for instance, ester, amide, carbamate, carbonate, cyclic ketal, thioester, thioamide, thiocarbamate, thiocarbonate, xanthate, phosphate ester, sulfonate, or a sulfamate linkage) or may be a linking group (for instance a C₁-C₁₂ dialcohol, a C₁-C₁₂ hydroxylalkanoic acid, a C₁-C₁₂ hydroxyalkylamine, a C₁-C₁₂ diacid, a C₁-C₁₂ aminoacid, or a C₁-C₁₂ diamine). Especially preferred linkages are direct amide, ester, carbonate, carbamate, and sulfamate linkages, and linkages via succinic acid, salicylic acid, diglycolic acid, oxa acids, oxamethylene, and halides thereof. The linkages are labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the codrug has not been solubilized in a physiologic fluid. Thus, while some codrugs according to the present invention may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g., non-aqueous solvents such as acetone). Thus, the labile linkages are such that, when the codrug is dissolved in an aqueous solution, especially a physiologic fluid such as may be found in the dermis or viable epidermis, the reaction is driven to the hydrolysis products, which include the constituent moieties set forth above.

In some embodiments according to the present invention, the first and second constituent pharmaceutically active moieties are each hydrocortisone.

In some embodiments according to the present invention, the first constituent moiety is an antiinflammatory compound and the second constituent moiety is an antipsoriatic compound. In specific embodiments according to the present invention, the first constituent moiety is a corticosteroid compound and the second constituent moiety is an antipsoriatic compound. In other embodiments according to the present invention, the first constituent moiety is an NSAID and the second constituent moiety is an antipsoriatic compound. In some embodiments according to the present invention, the first constituent moiety is an antiinflammatory compound and the second constituent moiety is an antibiotic compound.

An illustrative codrug according to the present invention for treatment of inflammation is a corticosteroid-corticosteroid codrug, such as a hydrocortisone-hydrocortisone codrug, specifically the hydrocortisone-maleate-hydrocortisone depicted below.

Codrug 1:

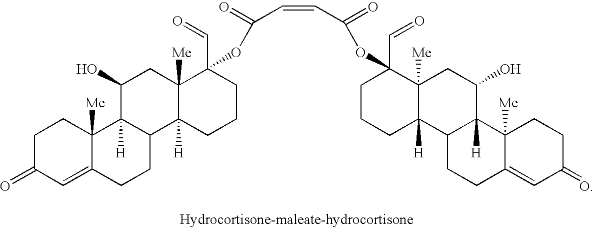

An illustrative codrug according to the present invention for the treatment of inflammation is an antipsoriatic-antiinflammatory codrug, such as the antipsoriatic-hydrocortisone codrug depicted below.

Codrug 2:

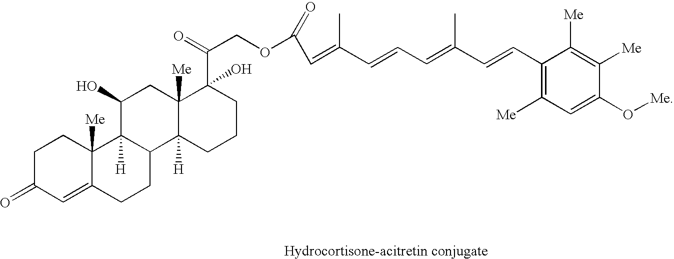

Codrugs for preparation of topical or transdermal compositions according to the present invention may be synthesized in the manner illustrated in one of the synthetic schemes below. In general, where the first and second pharmaceutically active moieties are to be directly linked, the first moiety is condensed with the second moiety under conditions suitable for forming a linkage that is labile under physiologic conditions. In some cases it is necessary to block some reactive groups on one, the other, or both of the moieties. Where the pharmaceutically active moieties are to be covalently linked via a linker, such as oxamethylene, succinic acid, or diglycolic acid, it is advantageous to first condense the first pharmaceutically active moiety with the linker. In some cases it is advantageous to perform the reaction in a suitable solvent, such as acetonitrile, in the presence of suitable catalysts, such as carbodiimides including EDCI (1-ethyl-3-(3-dimethylaminopropyl)-carbodiimide) and DCC (DCC: dicyclohexylcarbodiimide), or under conditions suitable to drive off water of condensation or other reaction products (e.g., reflux), or a combination of two or more thereof. After the first pharmaceutically active moiety is condensed with the linker, the combined first moiety and linker may then be condensed with the second pharmaceutically active moiety. Again, in some cases it is advantageous to perform the reaction in a suitable solvent, such as acetonitrile, in the presence of suitable catalysts, such as carbodiimides including EDCI and DCC, or under conditions suitable to drive off water of condensation or other reaction products (e.g., reflux), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

The person having skill in the art will recognize that, while diacids, dialcohols, amino acids, etc. are described above as being suitable linkers, other linkers are contemplated as being within the present invention. For instance, while the hydrolysis product of a codrug according to the present invention may comprise a diacid, the actual reagent used to make the linkage may be, for example, an acylhalide such as succinyl chloride. The person having skill in the art will recognize that other possible acid, alcohol, amino, sulfato, and sulfamoyl derivatives may be used as reagents to make the corresponding linkage.

Where the first and second pharmaceutically active moieties are to be directly linked via a covalent bond, essentially the same process is conducted, except that in this case there is no need for a step of adding a linker. The first pharmaceutically active moiety and second pharmaceutically active moieties are merely combined under conditions suitable for forming the covalent bond. In some cases it may be desirable to block certain active groups on one, the other, or both of the pharmaceutically active moieties. In some cases it may be desirable to use a suitable solvent, such as acetonitrile, a catalyst suitable to form the direct bond, such as carbodiimides including EDCI and DCC, or conditions designed to drive off water of condensation (e.g., reflux) or other reaction by-products.

The person having skill in the art will recognize that, while in most cases the first and second moieties may be directly linked in their original form, it is possible for the active groups to be derivatized to increase their reactivity. For instance, where the first moiety is an acid and the second moiety is an alcohol (i.e., has a free hydroxyl group), the first moiety may be derivatized to form the corresponding acid halide, such as an acid chloride or an acid bromide. The person having skill in the art will recognize that other possibilities exist for increasing yield, lowering production costs, improving purity, etc. of the codrug according to the present invention by using conventionally derivatized starting materials to make codrugs according to the present invention.

Exemplary reaction schemes according to the present invention are illustrated in Schemes 1-4, below. These Schemes can be generalized by substituting other therapeutic agents having at least one functional group that can form a covalent bond to another therapeutic agent having a similar or different functional group, either directly or indirectly through a pharmaceutically acceptable linker. The person of skill in the art will appreciate that these schemes also may be generalized by using other appropriate linkers.

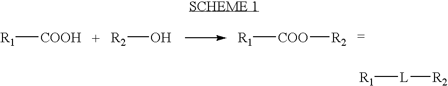

wherein L is an ester linker —COO—, and R₁ and R₂ are the residues of the first and second constituent moieties or pharmacological moieties, respectively.

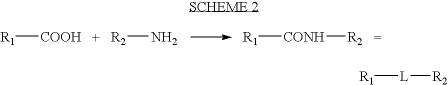

wherein L is the amide linker —CONH—, and R₁ and R₂ have the meanings given above.

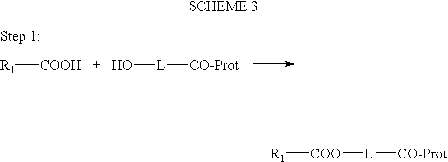

wherein Prot is a suitable reversible protecting group.

Step 2: R₁—COO-L-CO-Prot→R₁—COO-L-COOH

Step 3: R₁—COO-L-COOH+R₂—OH→R₁—COO-L-COOR₂

wherein R₁, L, and R₂ have the meanings set forth above.

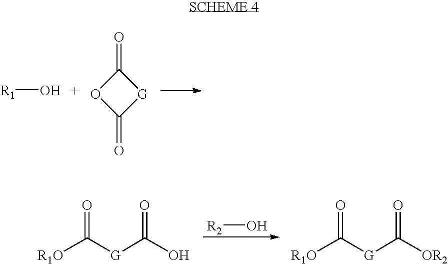

wherein R₁ and R₂ have the meanings set forth above and G is a direct bond, an C₁-C₄ alkylene, a C₂-C₄ alkenylene, a C₂-C₄ alkynylene, or a 1,2-fused ring, and G together with the anhydride group completes a cyclic anhydride. Suitable anhydrides include succinic anhydride, glutaric anhydride, malefic anhydride, diglycolic anhydride, and phthalic anhydride.

The factors influencing transdermal permeability of the stratum corneum can be classified into three major categories: 1) physicochemical properties of the penetrant; 2) physicochemical properties of the drug delivery system; and 3) physiological and pathological conditions of the skin. Various models based upon physicochemical data have been proposed to predict the transdermal flux of pharmaceutically active agents. Such models correlate the permeability of the skin to a specific penetrant (Kₚ) with the diffusion coefficient of the penetrant through the skin (Dₛ) and partition coefficient (kₘ) between the lipophilic stratum corneum and more aqueous in nature viable tissue.

Certain codrugs of the present invention are highly lipophilic, i.e., have log P values of greater than 3. Such a codrug may be expected to be readily absorbed by or into the stratum corneum, and remain in the stratum corneum due to its high lipophilicity, where a reservoir or depot of the codrug will form. This reservoir or depot may provide a sustained release delivery of the constituent moieties into the viable epidermis as the codrug leaches from the stratum corneum or is hydrolyzed into its constituent moieties, at least one of which may be less lipophilic than the codrug.

It has also been concluded that one would expect the specific permeability of skin to the non-ionized form of a compound to be substantially greater that that of the ionized form. However, if the water solubility of the free base, non-ionized form of the compound is much less than that of its ionized salt, its rate of permeation in non-ionized form may be lower than that of its salt, even though the intrinsic permeability of the skin for the free base may be much greater. See Y. W. Chien, Transdermal Controlled-Release Drug Administration, Novel Drug Delivery systems—Fundamentals, Development Concepts, Biomedical Assessments, Marcel Dekker Inc., N.Y. 1982; and Guy et al., Physicochemical Aspects of Percutaneous Penetration and Its Enhancement, Pharmaceutical Research, Vol. 5, No. 12, 1988, which are hereby incorporated in their entirety by reference.

II. Definitions

The term “active” as used herein means therapeutically or pharmacologically active.

The term “ED₅₀” means the dose of a biologically active moiety that produces 50% of its maximum response or effect.

The term “IC₅₀” means the dose of a biologically active moiety that inhibits a biological activity by 50%.

The term “LD₅₀” means the dose of a biologically active moiety that is lethal in 50% of test subjects.

The term “therapeutic index” refers to the therapeutic index of a biologically active moiety defined as LD₅₀/ED₅₀.

As used herein, the term “codrug” means a first constituent moiety chemically linked to at least one other constituent moiety that is the same as, or different from, the first constituent moiety. The individual constituent moieties are reconstituted as the pharmaceutically active forms of the same moieties, or prodrugs thereof, prior to conjugation.

As used herein, the term “constituent moiety” means one of two or more biologically active moieties so linked as to form a codrug according to the present invention as described herein. In some embodiments according to the present invention, two molecules of the same constituent moiety are combined to form a dimer. In the context where the free, unconjugated form of the moiety is referred to, the term “constituent moiety” means a pharmaceutically active moiety, either before it is combined with another pharmaceutically active moiety to form a codrug, or after the codrug has been hydrolyzed to remove the linkage between the two or more constituent moieties. In such cases, the constituent moieties are chemically the same as the pharmaceutically active forms of the same moieties, or prodrugs thereof, prior to conjugation.

“Improved dermal uptake” can be determined using in vitro skin permeation studies and in vivo skin tape-stripping methods. These methods are fully described in Brain et al., “Methods for studying percutaneous absorption,” in “Dermatological and Transdermal Formulations,” eds. K. Walters and M Dekker, NY, pp. 197-269, (2002).

“Log P” refers to the logarithm of P (Partition Coefficient). P is a measure of how well a substance partitions between octanol and water. P itself is a constant for a given molecule. It is defined as the ratio of concentration of compound in aqueous phase to the concentration of compound in an immiscible solvent, as the neutral molecule.
Partition Coefficient, P=[Organic]/[Aqueous] where []=concentration
Log P=log₁₀ (Partition Coefficient)=log₁₀ P

In practice, the Log P value will vary according to the conditions under which it is measured and the choice of partitioning solvent. A Log P value of 1 means that the concentration of the compound is ten times greater in the organic phase than in the aqueous phase. The increase in a log P value of 1 indicates a ten fold increase in the concentration of the compound in the organic phase as compared to the aqueous phase. Compounds with log P values greater than 5 are considered as having very low aqueous solubility. In general, compounds having log P values between 7 and 10 are considered almost insoluble in aqueous media.

In the context of referring to the codrug according to the present invention, the term “residue of a constituent moiety” means that part of a codrug that is structurally derived from a constituent moiety apart from the functional group through which the moiety is linked to another constituent moiety. For instance, where the functional group is —NH₂, and the constituent group forms an amide (—NH—CO—) bond with another constituent moiety, the residue of the constituent moiety is that part of the constituent moiety that includes the —NH— of the amide, but excluding the hydrogen (H) that is lost when the amide bond is formed. In this sense, the term “residue” as used herein is analogous to the sense of the word “residue” as used in peptide and protein chemistry to refer to a residue of an amino acid in a peptide.

The terms “drug” and “pharmaceutical” are interchangeable as used herein and have their art-recognized meanings.

As used herein, the phrase “the codrug is relatively lipophilic,” means that the codrug is more lipophilic than one or more of the constituent moieties that comprises it. In some embodiments according to the present invention, the codrug is more lipophilic than only one of the constituent moieties. In other embodiments according to the present invention, the codrug is more lipophilic than more than one of the constituent moieties, and in particular embodiments according to the present invention, the codrug is more lipophilic than all the constituent moieties of the codrug.

A “patient” or “subject” to be treated by the subject method can mean either a human or non-human animal.

A “pharmacological moiety” is a moiety that, when active or when activated, can cause an intended medical effect. Pharmacological moieties typically cause these effects when made to interact with a drug target (generally in the body of a subject to which the moiety has been administered, particularly a human or mammal that is a model of a human disease or condition, but possibly also in an animal, such as a bird or mammal, in a veterinary administration of the moiety).

The phrase “pharmaceutically acceptable carrier” as used herein means a pharmaceutically acceptable material, composition or vehicle, such as a liquid or solid filter, diluent, excipient, solvent or encapsulating material, involved in carrying or transporting the subject regulators from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) ethyl alcohol; (19) phosphate buffer solutions; and (20) other non-toxic compatible substances employed in pharmaceutical formulations.

“Physiological conditions” describe the conditions inside an organism, i.e., in vivo. Physiological conditions include the acidic and basic environments of body cavities and organs, enzymatic cleavage, metabolism, and other biological processes, and preferably refer to physiological conditions in a vertebrate, such as a mammal.

A “prodrug” is a moiety that is generally not pharmacologically active. However, when activated, typically in vivo by enzymatic or hydrolytic cleavage to convert the prodrug to an active biological moiety, the administration of the prodrug to the individual will have had the intended medical effect. Prodrugs are typically formed by chemical modification of a biologically active moiety. Prodrugs may also be used to increase transdermal absorption by enhancing permeation through topical membranes. Conventional procedures for the selection and preparation of suitable prodrug derivatives are described, for example, in Design of Prodrugs, ed. H. Bundgaard, Elsevier, 1985.

Throughout this application, the term “proliferative skin disorder” refers to any disease/disorder of the skin marked by unwanted or aberrant proliferation of cutaneous tissue. These conditions are typically characterized by epidermal cell proliferation or incomplete cell differentiation, and include, for example, X-linked ichthyosis, psoriasis, atopic dermatitis, allergic contact dermatitis, epidermolytic hyperkeratosis, and seborrheic dermatitis. For example, epidermodysplasia is a form of faulty development of the epidermis. Another example is “epidermolysis”, which refers to a loosened state of the epidermis with formation of blebs and bullae either spontaneously or at the site of trauma.

The term “carcinoma” refers to a malignant new growth made up of epithelial cells tending to infiltrate surrounding tissues and to give rise to metastases. Exemplary carcinomas include: “basal cell carcinoma”, which is an epithelial tumor of the skin that, while seldom metastasizing, has potentialities for local invasion and destruction; “squamous cell carcinoma”, which refers to carcinomas arising from squamous epithelium and having cuboid cells; “carcinosarcoma”, which include malignant tumors composed of carcinomatous and sarcomatous tissues; “adenocystic carcinoma”, carcinoma marked by cylinders or bands of hyaline or mucinous stroma separated or surrounded by nests or cords of small epithelial cells, occurring in the mammary and salivary glands, and mucous glands of the respiratory tract; “epidermoid carcinoma”, which refers to cancerous cells which tend to differentiate in the same way as those of the epidermis; i.e., they tend to form prickle cells and undergo comification; “nasopharyngeal carcinoma”, which refers to a malignant tumor arising in the epithelial lining of the space behind the nose; and “renal cell carcinoma”, which pertains to carcinoma of the renal parenchyma composed of tubular cells in varying arrangements. Another carcinomatous epithelial growth is “papillomas”, which refers to benign tumors derived from epithelium and having a papillomavirus as a causative agent; and “epidermoidomas”, which refers to a cerebral or meningeal tumor formed by inclusion of ectodermal elements at the time of closure of the neural groove.

As used herein, the term “psoriasis” refers to a hyperproliferative skin disorder which alters the skin's regulatory mechanisms. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the dermis layer and polymorphonuclear leukocyte infiltration into the epidermis layer resulting in an increase in the basal cell cycle. Additionally, hyperkeratotic and parakeratotic cells are present.

The term “keratosis” refers to proliferative skin disorder characterized by hyperplasia of the horny layer of the epidermis. Exemplary keratotic disorders include keratosis follicularis, keratosis palmaris et plantaris, keratosis pharyngea, keratosis pilaris, and actinic keratosis.

The term “skin” refers to the outer protective covering of the body, consisting of the corium and the epidermis, and is understood to include sweat and sebaceous glands, as well as hair follicle structures. Throughout the present application, the adjective “cutaneous” may be used, and should be understood to refer generally to attributes of the skin, as appropriate to the context in which they are used.

The term “epidermis” refers to the outermost and nonvascular layer of the skin, derived from the embryonic ectoderm, varying in thickness from 0.07-1.4 mm. On the palmar and plantar surfaces it comprises, from within outward, five layers: basal layer composed of columnar cells arranged perpendicularly; prickle-cell or spinous layer composed of flattened polyhedral cells with short processes or spines; granular layer composed of flattened granular cells; clear layer composed of several layers of clear, transparent cells in which the nuclei are indistinct or absent; and horny layer composed of flattened, cornified non-nucleated cells. In the epidermis of the general body surface, the clear layer is usually absent.

The “corium” or “dermis” refers to the layer of the skin deep to the epidermis, consisting of a dense bed of vascular connective tissue, and containing the nerves and terminal organs of sensation. The hair roots, and sebaceous and sweat glands are structures of the epidermis which are deeply embedded in the dermis.

The phrases “systemic administration,” “administered systemically,” “peripheral administration” and “administered peripherally” as used herein mean the administration of a biologically active moiety, codrug, or other material other than directly into the central nervous system, such that it enters the patient's system and, thus, is subject to metabolism and other like processes, for example, subcutaneous administration.

By “transdermal” is meant transdermal or percutaneous administration, i.e., application of the skin composition directly to the skin to be treated for systemic delivery of the codrugs of the invention. By “dermal” is meant local application of the codrug for topical delivery of the codrugs of the invention. In general the terms “skin,” “derma,” “epidermis,” and “dermis” are used interchangeably unless specifically stated otherwise.

By “transdermal patch” is meant a system capable of delivery of a biologically active moiety to a patient via the skin, or any suitable external surface, including mucosal membranes, such as those found inside the mouth. Such delivery systems generally comprise a flexible backing, an adhesive and a biologically active moiety retaining matrix, the backing protecting the adhesive and matrix and the adhesive holding the whole on the skin of the patient. On contact with the skin, the biologically active moiety-retaining matrix delivers a biologically active moiety to the skin, the moiety then passing through the skin into the patient's system.

The term “treatment” is intended to encompass also prophylaxis, therapy and cure. The patient receiving this treatment is any animal in need, including primates, in particular humans, and other mammals such as equines, cattle, swine and sheep; and poultry and pets in general.

A “substitution” or “substituent” on a small organic molecule generally refers to a valency on a multivalent atom occupied by a moiety other than hydrogen, e.g., a position on a chain or ring exclusive of the member atoms of the chain or ring. Such moieties include those defined herein and others as known in the art, for example, halogen, alkyl, alkenyl, alkynyl, azide, haloalkyl, hydroxyl, carbonyl (such as carboxyl, alkoxycarbonyl, formyl, ketone, or acyl), thiocarbonyl (such as thioester, thioacetate, or thioformate), alkoxyl, phosphoryl, phosphonate, phosphinate, amine, amide, amidine, imine, cyano, nitro, azido, sulfhydryl, alkylthio, sulfate, sulfonate, sulfamoyl, sulfonamido, sulfonyl, silyl, ether, cycloalkyl, heterocyclyl, heteroalkyl, heteroalkenyl, and heteroalkynyl, heteroaralkyl, aralkyl, aryl or heteroaryl. It will be understood by those skilled in the art that certain substituents, such as aryl, heteroaryl, polycyclyl, alkoxy, alkylamino, alkyl, cycloalkyl, heterocyclyl, alkenyl, alkynyl, heteroalkyl, heteroalkenyl, and heteroalkynyl, can themselves be substituted, if appropriate. This invention is not intended to be limited in any manner by the permissible substituents of organic compounds. It will be understood that ‘substitution’ or ‘substituted with’ includes the implicit proviso that such substitution is in accordance with permitted valence of the substituted atom and the substituent, and that the substitution results in a stable compound, e.g., which does not spontaneously undergo transformation such as by rearrangement, cyclization, elimination, hydrolysis, etc.

The terms ‘amine’ and ‘amino’ are art-recognized and refer to both unsubstituted and substituted amines as well as ammonium salts, e.g., as can be represented by the general formula: 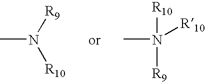
wherein R₉, R₁₀, and R′₁₀ each independently represent hydrogen or a hydrocarbon substituent, or R₉ and R₁₀ taken together with the N atom to which they are attached complete a heterocycle having from 4 to 8 atoms in the ring structure. In preferred embodiments, none of R₉, R₁₀, and R′₁₀ is acyl, e.g., R₉, R₁₀, and R′₁₀ are selected from hydrogen, alkyl, heteroalkyl, aryl, heteroaryl, carbocyclic aliphatic, and heterocyclic aliphatic. The term ‘alkylamine’ as used herein means an amine group, as defined above, having at least one substituted or unsubstituted alkyl attached thereto. Amino groups that are positively charged (e.g., R′₁₀ is present) are referred to as ‘ammonium’ groups. In amino groups other than ammonium groups, the amine is preferably basic, e.g., its conjugate acid has a pKₐ above 7.

The terms ‘amido’ and ‘amide’ are art-recognized as an amino-substituted carbonyl, such as a moiety that can be represented by the general formula: 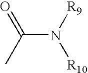
wherein R₉ and R₁₀ are as defined above. In certain embodiments, the amide will include imides. In general, when the oxygen of the above formula is replace by sulfur, the formula represents a ‘thioamide’.

The term ‘carbonyl’ is art-recognized and includes such moieties as can be represented by the general formula: 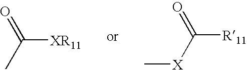
wherein X is a bond or represents an oxygen or a sulfur, and R₁₁ represents a hydrogen, hydrocarbon substituent, or a pharmaceutically acceptable salt, R₁₁, represents a hydrogen or hydrocarbon substituent. Where X is an oxygen and R₁₁ or R₁₁, is not hydrogen, the formula represents an ‘ester’. Where X is an oxygen, and R₁₁ is as defined above, the moiety is referred to herein as a carboxyl group, and particularly when R₁₁ is a hydrogen, the formula represents a ‘carboxylic acid’. Where X is an oxygen, and R ₁₁, is hydrogen, the formula represents a ‘formate’. In general, where the oxygen atom of the above formula is replaced by sulfur, the formula represents a ‘thiocarbonyl’ group. Where X is a sulfur and R₁₁ or R₁₁, is not hydrogen, the formula represents a ‘thioester.’ Where X is a sulfur and R₁₁ is hydrogen, the formula represents a ‘thiocarboxylic acid.’ Where X is a sulfur and R₁₁, is hydrogen, the formula represents a ‘thioformate.’ On the other hand, where X is a bond, R₁₁ is not hydrogen, and the carbonyl is bound to a hydrocarbon, the above formula represents a ‘ketone’ group. Where X is a bond, R₁₁ is hydrogen, and the carbonyl is bound to a hydrocarbon, the above formula represents an ‘aldehyde’ or ‘formyl’ group.

‘Carbamate’ refers to the group having the following general structure 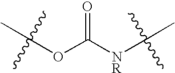
wherein R represents hydrogen or a hydrocarbon substituent.

A ‘thiocarbamate’ refers to a variant of the above group wherein the oxygen of the carbonyl is replaced by sulfur.

‘Carbonate’ refers to the group having the following general structure of

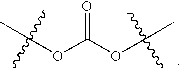

A ‘thiocarbonate’ refers to a variant of the above structure wherein the oxygen of the carbonyl is replaced by sulfur.

‘Cyclic ketal’ refers to a cyclic aliphatic group including two oxygen atoms, such as moieties having one of the following general structures: 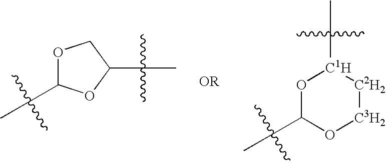
wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

‘Phosphate ester’ has refers to a group having the following general structure 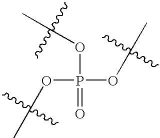
wherein each of the groups attached to the oxygens may be hydrogen, hydrocarbon, or a counterion (such as sodium) or other substituents as defined above.

A cyclic phosphate ester has the following general structure 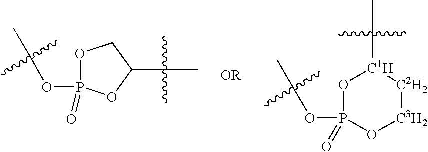
wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

‘Guanidino’ refers to a group having the following general structure 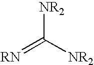
wherein each R may be, independently for each occurrence, a hydrogen or a hydrocarbon substituent. Two R's taken together may form a ring. The general structure may thus be part of one ring or a polycyclic structure.

‘Amidines’ are represented by the general formula 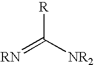
and are basic groups wherein each R may be, independently for each occurrence, a hydrogen or a hydrocarbon substituent. Two R taken together may form a ring.

‘Hydrocarbon substituents’ are moieties that include at least one C—H bond, and include groups such as alkyl, heteroalkyl, aryl, heteroaryl, carbocyclic aliphatic, and heterocyclic aliphatic groups.

‘Heteroatom’ refers to a multivalent non-carbon atom, such as a boron, phosphorous, silicon, nitrogen, sulfur, or oxygen atom, preferably a nitrogen, sulfur, or oxygen atom. Groups containing more than one heteroatom may contain different heteroatoms.

‘Heterocyclic aliphatic ring’ is a non-aromatic saturated or unsaturated ring containing carbon and from 1 to about 4 heteroatoms in the ring, wherein no two heteroatoms are adjacent in the ring and preferably no carbon in the ring attached to a heteroatom also has a hydroxyl, amino, or thiol group attached to it. Heterocyclic aliphatic rings are monocyclic, or are fused or bridged bicyclic ring systems. Monocyclic heterocyclic aliphatic rings contain from about 4 to about 10 member atoms (carbon and heteroatoms), preferably from 4 to 7, and most preferably from 5 to 6 member atoms in the ring. Bicyclic heterocyclic aliphatic rings contain from 8 to 12 member atoms, preferably 9 or 10 member atoms in the ring. Heterocyclic aliphatic rings may be unsubstituted or substituted with from 1 to about 4 substituents on the ring. Preferred heterocyclic aliphatic ring substituents include halo, cyano, lower alkyl, heteroalkyl, haloalkyl, phenyl, phenoxy or any combination thereof. More preferred substituents include halo and haloalkyl. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, hydantoin, oxazoline, imidazolinetrione, triazolinone, quinoline, phthalazine, naphthyridine, quinoxaline, quinazoline, quinoline, pteridine, carbazole, carboline, pbenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Preferred heterocyclic aliphatic rings include piperazyl, morpholinyl, tetrahydrofuranyl, tetrahydropyranyl and piperidyl. Heterocycles can also be polycycles.

‘Heteroalkyl’ is a saturated or unsaturated chain of carbon atoms and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl have one or two branches, preferably one branch. Preferred heteroalkyl are saturated. Unsaturated heteroalkyl have one or more double bonds and/or one or more triple bonds. Preferred unsaturated heteroalkyl have one or two double bonds or one triple bond, more preferably one double bond. Heteroalkyl chains may be unsubstituted or substituted with from 1 to about 4 substituents unless otherwise specified. Preferred heteroalkyl are unsubstituted. Preferred heteroalkyl substituents include halo, aryl (e.g., phenyl, tolyl, alkoxyphenyl, alkoxycarbonylphenyl, halophenyl), heterocyclyl, heteroaryl. For example, alkyl chains substituted with the following substituents are heteroalkyl: alkoxy (e.g., methoxy, ethoxy, propoxy, butoxy, pentoxy), aryloxy (e.g., phenoxy, chlorophenoxy, tolyloxy, methoxyphenoxy, benzyloxy, alkoxycarbonylphenoxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkoxycarbonylphenylthio), amino (e.g., amino, mono- and di-C1-C3 alkylamino, methylphenylamino, methylbenzylamino, C1-C3 alkylamido, carbamamido, ureido, guanidino).

“Phannaceutically acceptable salt” refers to a cationic salt formed at any acidic (e.g., hydroxamic or carboxylic acid) group, or an anionic salt formed at any basic (e.g., amino or guanidino) group. Such salts are well known in the art. See e.g., PCT Publication 87/05297, incorporated herein by reference. Such salts are made by methods known to one of ordinary skill in the art. It is recognized that the skilled artisan may prefer one salt over another for improved solubility, stability, formulation ease, price and the like. Determination and optimization of such salts is within the purview of the skilled artisan's practice. Preferred anions include halides (such as chloride), sulfonates, carboxylates, phosphates, therapeutically active carboxylates, and the like.

A “xanthate” refers to the group having the following general structure 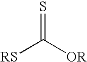
wherein R represents a hydrocarbon substituent.
III. Exemplary Codrugs

In some embodiments, the codrugs of the invention are formed by covalent conjugation of two or more constituent moieties. The constituent moieties can be linked to form a single codrug by reversible covalent bonds such that, at the desired site in the body, the covalently-linked constituent moieties are cleaved to regenerate the active forms of the constituent moieties, or the prodrug precursors to the biologically active moieties of interest. The rate of cleavage of the constituent moieties can be controlled by the type of the bond linking the constituent moieties, the choice of constituent moieties, and the physical form of the moieties. Codrugs according to the present invention are labile in water, serum, or other bodily fluids, and regenerate the biologically active moieties or prodrugs thereof. In some embodiments, the codrugs of the present invention have very low solubility in one or more of serum and other bodily fluids, and are quickly hydrolyzed to regenerate the biologically active moieties or prodrugs thereof upon dissolution in a biological environment.

Each constituent moiety possesses one or more functional groups that are capable of forming a labile bond with another constituent moiety, or with a linkage that is linked to a constituent moiety. Suitable labile bonds include ester, amide, carbamate, carbonate, cyclic ketal, thioester, thioamide, thiocarbamate, thiocarbonate, xanthate, phosphate ester, sulfonate, or a sulfamate, anhydride, urea, guanidino, and sulfonamido bonds. Suitable functional groups for forming these bonds include amino, carboxylic acid, hydroxy, thiol, and sulfonate groups. Suitable linking groups include diacids, diamines, amino acids, hydroxy acids, hydroxy amines, dialcohols, etc.

The constituent moieties may be any biologically active moieties that possess one or more functional groups that may form hydrolyzable bonds with themselves (e.g., dimers, trimers, etc.), other biologically active moieties, or with a linkage if one is used. The constituent moieties may be, for instance, antidepressant compounds; analgesic compounds such as lidocaine, benzodiazepam, tramadol, and related compounds; anti-inflammatory steroidal compounds (corticosteroids); non-steroidal antiinflammatory compounds (NSAIDs) such as diclofenac, naproxen, ketorolac, flurbiprofen, and indomethacin; antibiotic compounds; anti-fungal compounds such as fluconazole and related compounds; antiviral compounds such as foscamet sodium, trifluorothymidine, acyclovir, ganciclovir, dideoxyinosine (ddI), dideoxycytidine (ddC); antiproliferative compounds such as 5-fluorouracil, adriamycin and related compounds; antiglaucoma compounds such as carbonic anhydrase inhibitors, beta blockers, miotics, cholinesterase inhibitors, and sympathomimetics; immunomodulatory compounds such as muramyl dipeptide and related compounds; cell transport/mobility impeding agents such as colchicine, vincristine, cytochalsian B, and related compounds; cytokines and peptides/proteins such as cyclosporin, insulin, growth factor or growth hormones; skin-treating compounds such as vitamin E or retinol; sunscreens; skin protectants; antimetabolite compounds; antipsoriatic compounds; keratolytic compounds; anxiolytic compounds; antipsychotic compounds; etc.

Antiproliferative agents that are suitable for R₁ possess one or more functional groups that may react with either a functional group on R₂ or a linkage to form a bond. Exemplary functional groups possessed by R₁ include hydroxy groups, amine groups, carboxylate groups (including carboxylic acids and esters), acid anhydride groups, thiol groups, sulfonyl halide groups, etc. Preferred functional groups are —OH, —NH₂, —CO₂H, and —CO₂⁻ groups (where the dash indicates bonding to the residue of the antiproliferative compound).

Antiproliferative compounds suitable as one or more constituent moieties in the present invention include: alitretinoin(9-cis-retinoic acid); amifostine; bexarotene(4-[1-(5,6,7,8-tetrahydro-3,5,5,8,8-pentamethyl-2-naphthalenyl)ethenyl]benzoic acid); bleomycin; capecitabine(5′-deoxy-5-fluoro-cytidine); chlorambucil; cladribine; cytarabine; daunorubicin; docetaxel; doxorubicin; epirubicin; estramustine; etoposide; exemestane(6-methylenandrosta-1,4-diene-3,17-dione); fludarabine; 5-fluorouracil; gemcitabine; hydroxyurea; idarubicin; irinotecan; melphalan; methotrexate; mitoxantrone; paclitaxel; pentostatin; prednimustine; streptozocin; temozolamide; teniposide; tomudex; topotecan; valrubicin(N-trifluoroacetyladriamycin-14-valerate); vinorelbine; and salts of the foregoing. Preferred antiproliferative agents are paclitaxel, docetaxel, methotrexate, and 5-fluorouracil. Each of these antiproliferative compounds possesses one or more functional groups as defined above, and all are thus capable of being linked to one or more of the same antiproliferative compound, a different antiproliferative compound, or a different pharmaceutically active compound, having a similar or different functional group, either directly or indirectly through a pharmaceutically acceptable linker.

Suitable corticosteroids for use as one or more constituent moieties according to the present invention include: 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide. Each of these corticosteroid moieties possesses one or more functional groups as defined above, and all are thus capable of being linked to one or more of the same corticosteroid, a different corticosteroid, or a different pharmaceutically active moiety.

Preferred corticosteroid moieties for preparing codrugs according to the present invention include moieties of the formula:

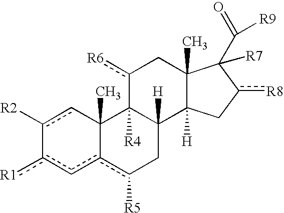

wherein R1 is ═O, —OH, or —(CH₂)₁₋₄Cl;

R2 is H, C₁₋₄alkyl, Cl, or Br;

R4 is H, F, or Cl;

R5 is H, F, Cl, CH₃, or —CHO;

R6 is H, OH, or Cl;

R7 is H, OH, CH₃, O—COCH₃, O(CO)OCH₂CH₃, O—(CO)-2-furanyl, or O—C(O)—(CH₂)₂CH₃;

R8 is H, CH₃, OH, ═CH₂, or together R7 and R8 form, together with the adjacent carbon atoms to which they are attached:

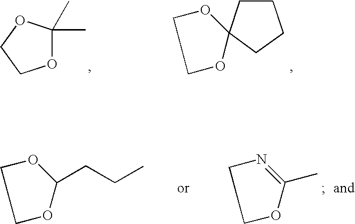

R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)-NH(CO)C₆H₅, or —S—CH₂—F; and

wherein the bonds indicated by

[symbol]

are either double or single bonds.

One skilled in the art will recognize that the class of corticosteroid compounds is a distinct class of steroids that does not include estrogens or androgens.

Illustrative examples of suitable β-lactam antibiotics include, amoxicillin, ampicillin, amylpenicillin, apalcillin, azidocillin, azlocillin, aztreonam, bacampicillin, benzylpenicillinic acid, biapenem, cefaclor, cefadroxil, cefamandole, cefatrizine, cefazedone, cefazolin, cefbuperazone, cefcapene pivoxil, cefclidin, cefdinir, cefditoren, cefepime, cefetamet, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsolodin, ceftazidime, cefteram, ceftezole, ceftibuten, ceftiofur, ceftizoxime, ceftriaxone, cefuroxime, cefuzonam, cephacetrilic acid, cephalexin, cephaloglycin, cephaloridine, cephalosporin C, cephalothin, cephamycins, cephapirinic acid, cephradine, clometocillin, cloxacillin, cyclacillin, dicloxacillin, fenbenicillin, flomoxef, floxacillin, hetacillin, imipenem, lenampicillin, loracarbef, meropenem, metampicillin, moxalactam, norcardicins (e.g., norcardicin A), oxacillin, panipenem, penicillin G, penicillin N, penicillin O, penicillin S, penicillin V, phenethicillin, piperacillin, pivampicillin, pivcefalexin, propicillin, sulbenicillin, sultamicillin, talampicillin, temocillin, ticarcillin, and tigemonam. Each of the above-identified β-lactam antibiotics possesses at least one functional group capable of forming a covalent bond to at least one other pharmaceutically effective moiety having at least one functional group, either directly or via a labile linker.

Antibiotic compounds suitable as one of more constituent moieties in the present invention include: metronidazole, ciprofloxacin, etc.

Non-steroidal anti-inflammatory (NSAID) compounds that are suitable for R₂ possess one or more functional groups that may react with either a functional group on R₁ or a linkage to form a bond. Exemplary functional groups possessed by R₂ include hydroxy groups, amine groups, carboxylate groups (including carboxylic acids and esters), acid anhydride groups, thiol groups, sulfonyl halide groups, etc. Preferred functional groups are —OH, —NH₂, —CO₂H (including —CO₂⁻) groups, (the dashes indicating bonding to the residue of the antiproliferative compound).

NSAID compounds suitable as one or more constituent moieties in the present invention include: diclofenac, etodolac, indomethacin, sulindac, tolmetin, nabumetone, piroxicam, acetaminophen, aspirin, fenoprofen, flurbiprofen, ibuprofen, ketorolac, ketoprofen, naproxen, oxaprozin, choline magnesium trisalicylate, diflunisal, meclofenamic acid, mefenamic acid, and phenylbutazone, or prodrugs, salts, or active metabolites thereof. Each of the foregoing NSAID compounds possesses at least one functional group capable of forming a direct or indirect bond to another moiety having one or more functional groups, and all are thus capable of being linked to one or more of the same NSAID, a different NSAID, or a different pharmaceutically active moiety. Preferred NSAIDs for making codrugs according to the present invention are diclofenac, flurbiprofen, naproxen, and ketoprofen. Preferred salts include sodium and potassium salts.

Suitable analgesic compounds for use as one or more constituent moieties according to the present invention include: benzodiazepam, buprenorphine, butorphanol, codeine, desmorphine, dezocine, dihydromorphine, dimepbeptanol, eptazocine, ethylmorphine, fentanyl, glafenine, hydromorphone, isoladol, ketobenidone, p-lactophetide, levorphanol, lidocaine, moptazinol, metazocin, meperidine, methadone, metopon, morphine, nalbuphine, nalmefene, nalorphine, naloxone, norlevorphanol, normorphine, oxycodone, oxymorphone, pentazocine, phenperidine, phenylramidol, propoxyphene, tramadol, and viminol, and salts and pharmaceutically esters and pro-drugs thereof. Each of these analgesic compounds above possesses one or more functional groups as defined above, and all are analgesics capable of being linked to one or more of the same analgesic, a different analgesic, or a different pharmaceutically active moiety.

Antidepressants that may be used as parent moieties in the present invention include bicyclic antidepressants, such as caroxazone, fencamine, indalpine, indeloxazine, nomifensine, oxitriptan (L-5HTP), paroxetine, and sertraline; hydrazides, such as benmoxine, iproclozide, iproniazid, isocarboxazid, octamoxin, and phenelzine; pyrrolidones, such as rolicyprine, rolipram, and sertindole; tetracyclic antidepressants, such as maprotiline; tricyclic antidepressants such as amoxapine, demexiptiline, desipramine, metapramine, nortiptaline, opipramol, propizepine, protriptyline, and tianeptine; and other antidepressants, such as adrafinil, benactyzine, dioxadrol, duloxetine, febarbamate, fenpentadiol, fluvoxamine, hematoporphyrin, hypericine, levophacetoperane, milnacipran, minaprine, moclobemide, pyrisuccideanol, roxindole, sulpiride, toloxatone, tranylcypromine, 1-tryptophan, venlafaxine, and viloxazine. Each of these antidepressant compounds possesses one or more functional groups as defined above, and all are thus capable of being linked to one or more of the same antidepressant compounds, a different antidepressant compound, or a different pharmaceutically active moiety.

Antipsychotic compounds that may be used as parent compounds in the present invention include benzamides, such as amisulpride, nemonapride, and sulpiride; benzisoxazoles; butyrophenones, such as benperidol, bromperidol, droperidol, haloperidol, moperone, pipamperone, spiperone, timiperone, and trifluperidol; phothiazines, such as acetophenazine, carphenazine, dixyrazine, fluphenazine, pericyazine, perimethazine, perphenazine, piperacetazine, and pipotiazine; thioxanthenes, such as clopenthixol and flupentixol; other tricyclic antipsychotic compounds, such as carpipramine, clocapramine, mosaprimine, olanzapine, opipramol, and seroquel; and other antipsychotics, such as buramate, penfluridol, pimozide, and ziprasidone. Each of these antipsychotic compounds possesses one or more functional groups as defined above, and all are thus capable of being linked to one or more of the same antipsychotic compound, a different antipsychotic compound, or a different pharmaceutically active moiety.

Anxiolytic compounds that may be used as parent compounds in the present invention include arylpiperazines, such as enciprazine and flesinoxan; benzodiazepine derivatives, such as chlordiazepoxide, clorazepate, flutazolam, lorazepam, mexazolam, nordazepam, and oxazepam; carbamates, such as emylcamate, hydroxyphenamate, meprobamate, phenprobamate, and tybamate; other anxiolytic compounds, such as benzoctamine, glutamic acid, hydroxyzine, mecloralurea, mephenoxalone, and oxanamide; and selective serotonin uptake inhibitors (SSRI's), such as fluoxetine, fluvoxamine, indalpine, indeloxazine HCl, milnacipran, paroxetine, and sertraline. Each of these anxiolytic compounds possesses one or more functional groups as defined above, and all are thus capable of being linked to one or more of the same anxiolytic compound, a different anxiolytic compound, or a different pharmaceutically active moiety.

Keratolytic compounds suitable as one or more constituent compounds in the present invention include: retinoic acid (vitamin A), dichloroacetic acid, resorcinol, salicylic acid, and tetroquinone. Each of these keratolytic compounds possesses one or more functional groups as defined above, and all are thus capable of being linked to one or more of the same keratolytic compound, a different keratolytic compound, or a different pharmaceutically active moiety.

Antimetabolite compounds interfere with the normal metabolic processes within cells, e.g., by combining with the enzymes responsible for them. Antimetabolite compounds suitable as one or more constituent compounds in the present invention include: 5-fluorouracil, methotrexate, 5-fluoro-2′-deoxyuridine (FUDR), Ara-C (cytarabine), gemcitabine, mercaptopurine, and other modified nucleotides and nucleosides. Each of these antimetabolite compounds possesses one or more functional groups as defined above, and all are thus capable of being linked to one or more of the same antimetabolite compound, a different antimetabolite compound, or a different pharmaceutically active moiety.Antipsoriatic compounds suitable as one or more constituent moieties in the present invention include: retinoids (including but not limited to retinoic acid, acitretin and tazarotene), salicylic acid (monoammonium salt), anthralin, 6-azauridine, vitamin D derivatives (including but not limited to calcipotriene and calcitriol), pyrogallol, and tacalcitol. Each of these antipsoriatic compounds possesses one or more functional groups as defined above, and all are thus capable of being linked to one or more of the same antipsoriatic compound, a different antipsoriatic compound, or a different pharmaceutically active moiety.

Exemplary sunscreens suitable as one or more constituent moieties in the present invention include: actinoquinol, p-aminobenzoic acid (PABA), and 4-dimethylaminobenzoic acid. Each of these sunscreen compounds possesses one or more functional groups as defined above, and all are thus capable of being linked to one or more of the same sunscreen compound, a different sunscreen compound, or a different pharmaceutically active moiety.

Exemplary skin protectants suitable as one or more constituent moieties in the present invention include: allantoin and esculin. Each of these skin protectant compounds possesses one or more functional groups as defined above, and all are thus capable of being linked to one or more of the same skin protectant compound, a different skin protectant compound, or a different pharmaceutically active moiety.

In particular embodiments according to the present invention, a therapeutically effective amount of a biologically active moiety, salt, or composition according to the present invention will deliver a systemic transdermal amount for a period of from about one day to ten days, preferably from about two days to a week. In certain other embodiments, the systemic transdermal amount is delivered in less than a day, e.g., in the course of a few hours or less.

In particular embodiments according to the present invention, a therapeutically effective amount of a biologically active moiety, salt, or composition according to the present invention will deliver a local dermal amount for a period of from about one day to ten days, preferably from about two days to a week. In certain other embodiments, local dermal amount is delivered in less than a day, e.g., in the course of a few hours or less.

In particular embodiments according to the present invention, a therapeutically effective amount of a biologically active moiety, salt, or composition according to the present invention will deliver a locally cytotoxic amount of an antiproliferative agent for a period of from about one day to ten days, preferably from about two days to a week. In certain other embodiments, locally cytotoxic amount is delivered in less than a day, e.g., in the course of a few hours or less.

In some embodiments according to the present invention, a therapeutically effective amount of a biologically active moiety, salt, or composition according to the present invention will deliver a locally apoptotic amount of an antiproliferative agent for a period of from about one day to ten days, preferably from about two days to a week. In certain other embodiments, locally apoptotic amount is delivered in less than a day, e.g., in the course of a few hours or less.

In some embodiments according to the present invention, a therapeutically effective amount of a biologically active moiety, salt, or composition according to the present invention will deliver a locally antiinflammatory amount of an antiproliferative agent for a period of from about one day to ten days, preferably from about two days to a week. In certain other embodiments, locally antiinflammatory amount is delivered in less than a day, e.g., in the course of a few hours or less.

The codrugs may be used for treating tumors in some embodiments. The codrugs release locally therapeutic levels of anti-neoplastic moieties while, at the same time, releasing locally effective levels of corticosteroid moieties. The codrugs thus treat tumors while simultaneously reducing the inflammation, and in some cases, the pain and/or stenosis associated with tumors. This dual action increases the efficacy of the codrugs by improving patient tolerance of the anti-neoplastic therapy. The dual action also may, in some cases, reduce diffusive efflux multiple drug resistance by reducing inflammation and the associated elevated fluid pressure in the vicinity of the tumor.

IV. Exemplary Methods

The pharmaceutical preparations of the invention may be useful for the treatment of hyperplastic epidermal conditions, such as keratosis, as well as for the treatment of neoplastic epidermal conditions such as those characterized by a high proliferation rate for various skin cancers, as for example basal cell carcinoma or squamous cell carcinoma. The subject method can also be used in the treatment of autoimmune diseases affecting the skin, in particular, of dermatological diseases involving morbid proliferation and/or keratinization of the epidermis, as for example, caused by psoriasis or atopic dermatosis.

Many common diseases of the skin, such as psoriasis, squamous cell carcinoma, keratoacanthoma and actinic keratosis are characterized by localized abnormal proliferation and growth. For example, in psoriasis, which is characterized by scaly, red, elevated plaques on the skin, the keratinocytes are known to proliferate much more rapidly than normal and to differentiate less completely.

In one embodiment, the preparations of the present invention are suitable for the treatment of dermatological ailments linked to keratinization disorders causing abnormal proliferation of skin cells, which disorders may be marked by inflammatory components. Psoriasis, as described above, is typically characterized by epidermal keratinocytes which display marked proliferative activation and differentiation along a “regenerative” pathway. Treatment with an antiproliferative embodiment of the subject method can be used to reverse the pathological epidermal activation and can provide a basis for sustained remission of the disease.

A variety of other keratotic lesions are also candidates for treatment with the subject antiproliferative preparations. Actinic keratoses, for example, are superficial inflammatory premalignant tumors arising on sun-exposed and irradiated skin. The lesions are erythematous to brown with variable scaling. Current therapies include excisional and cryosurgery. These treatments are painful, however, and often produce cosmetically unacceptable scarring. Accordingly, treatment of keratosis, such as actinic keratosis, can include application, preferably topical, of a composition of the present invention in amounts sufficient to inhibit hyperproliferation of epidermal/epidermoid cells of the lesion.

Acne represents yet another dermatologic ailment which may be treated with an antiproliferative embodiment of the subject method. Acne vulgaris, for instance, is a multifactorial disease most commonly occurring in teenagers and young adults, and is characterized by the appearance of inflammatory and noninflammatory lesions on the face and upper trunk. The basic defect which gives rise to acne vulgaris is hypercornification of the duct of a hyperactive sebaceous gland. Hypercomification blocks the normal mobility of skin and follicle microorganisms, and in so doing, stimulates the release of lipases by Propinobacterium acnes and Staphylococcus epidermidis bacteria and Pitrosporum ovale, a yeast. Treatment with an antiproliferative codrug of the present invention, particularly topical preparations, may be useful for preventing the transitional features of the ducts, e.g. hypercornification, which lead to lesion formation. The subject treatment may further include, for example, antibiotics, retinoids, and antiandrogens.

The present invention also provides a method for treating various forms of dermatitis. Dermatitis is a descriptive term referring to poorly demarcated lesions which are either pruritic, erythematous, scaly, blistered, weeping, fissured or crusted. These lesions arise from any of a wide variety of causes. The most common types of dermatitis are atopic, contact and diaper dermatitis. For instance, seborrheic dermatitis is a chronic, usually pruritic, dermatitis with erythema, dry, moist, or greasy scaling, and yellow crusted patches on various areas, especially the scalp, with exfoliation of an excessive amount of dry scales stasis dermatitis, an often chronic, usually eczematous dermatitis. Actinic dermatitis is dermatitis that due to exposure to actinic radiation such as that from the sun, ultraviolet waves or x- or gamma-radiation. According to the present invention, the subject codrug preparations can be used in the treatment and/or prevention of certain symptoms of dermatitis caused by unwanted proliferation of epithelial cells. Such therapies for these various forms of dermatitis can also include topical and systemic corticosteroids, antipruritics, and antibiotics.

In certain embodiments according to the present invention, the method comprises applying to the skin of a patient in need thereof a hydrocortisone-hydrocortisone codrug as described above. In particular embodiments of the invention, the hydrocortisone-hydrocortisone codrug is applied directly to the skin in the form of an ointment, salve, lotion, or cream, and the effect is to reduce local inflammation, itching, and/or pain.

In some embodiments according to the present invention, the method comprises administering an effective amount of a composition of the present invention for the treatment of psoriasis. In certain embodiments, the composition comprises a codrug comprising a first constituent moiety selected from corticosteroids and NSAIDs, and a second constituent moiety selected from antipsoriatic moieties, such as acitretin, salicylic acid, anthralin, 6-azauridine, calcipotriene, pyrogallol, and tacalcitol. In particular embodiments of the present invention, the method comprises using a composition containing a codrug of a corticosteroid, such as cortisone, hydrocortisone, prednisolone, and prednisone, and an antipsoriatic moiety, such as acitretin, antralin, or 6-azauridine.

The present invention also provides methods for treating a neoplastic disease. A method according to the present invention is useful for treating a cancerous or benign lesion, such as a solid tumor. Cancers treatable with one or more biologically active moieties according to the present invention include breast cancer, cervical cancer, uterine cancer, ovarian cancer, lung cancer, prostate cancer, liver cancer, pancreatic cancer, and lymphomas, including Hodgkins and non-Hodgkins lymphomas. Other neoplastic diseases treatable with codrugs according to the present invention include benign prostatic hyperplasia (BPH). A preferred method of treatment according to the present invention is treatment of BPH or prostate cancer, optionally in combination therapy with radiotherapy.

The method comprises administering to an individual, such as a human or non-human mammal, at least one therapeutically effective dose of a codrug, a salt thereof, or a composition comprising a codrug. A therapeutically effective amount of a codrug, salt, or composition according to the present invention is an amount that, when administered in a course of treatment, is able to bring about one or more of the following effects: halt the growth or spread of a neoplastic disease, prevent metastasis of a neoplastic lesion, produce a cytotoxic effect in a neoplastic lesion, induce apoptosis in cancerous or pre-cancerous neoplastic cells, reduce or prevent local or systemic inflammation, or reduce pain associated with a neoplastic lesion. In certain embodiments according to the present invention, a therapeutically effective dose is an amount of a codrug, salt, or composition according to the present invention that releases sufficient antiproliferative agent in sufficient concentration over a period of time sufficient to produce a cytotoxic effect in the target neoplastic lesion.

A method according to the present invention advantageously employs a codrug or a composition according to the present invention via topical administration.

The present invention includes methods for treatment of a patient in need of such treatment. The patient may be of any mammalian species, especially human. Veterinary patients include species of dogs, cats, horses, cattle, and swine. The need for treatment is determined by a skilled physician or veterinarian based upon the symptoms presented by the patient.

The method for treatment of a patient in need of such treatment comprises applying a pharmaceutically effective amount of a composition according to the present invention, which comprises a codrug as described herein, to the skin. The composition may be applied directly to the skin, in the form of an ointment, salve, lotion, cream, gel, or other physical form suitable for application to the skin. The composition may also be applied indirectly to the skin in the form of a medical device as discussed herein, and in particular as a microneedle, a bandage, gauze pad, or patch as described herein.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the constituent drugs of the particular codrug of the present invention employed, or the ester, salt, or amide thereof, the route of administration, the time of administration, the rate of excretion of the particular codrug (and/or its constituent drugs) being employed, the duration of the treatment, other biologically active moieties, materials used in combination with the particular codrug employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the codrugs of the invention employed in the pharmaceutical composition at levels lower than that required in order to achieve the desired therapeutic effect and gradually increase the dosage until the desired effect is achieved.

The skilled physician or veterinarian will recognize that a method of treatment requires application of a pharmaceutically effective amount of the codrug to the patient's skin. A pharmaceutically effective amount of the codrug will vary from patient to patient, and will be determined in part by the patient's species, sex, body mass, age, and general health. The effective amount will also depend upon the dermal permeability of the codrug, whether permeability enhancers are included in the pharmaceutical composition, whether the intended effect is systemic or topical, etc. Where a systemic effect is intended, the amount of codrug applied to the skin in a single dose should be such as to release approximately 0.001 mg to 100.0 mg per kilogram of the patient's body mass per day. Where a topical effect is sought, the amount of codrug should be adjusted in proportion to the surface area of the site to be treated as it relates to the total surface area of the body. Illustrative doses for topical administration are in the range of about 0.001 μg to 100 μg per cm² to be treated.

The method of treatment according to the present invention can be used to treat a number of diverse physical ailments. In this context, the terms treat, treating, and treatment include alleviation of one or more symptoms, reduction in the rate of progress of a progressive disease state, induction of remission of a disease state, and cure. In some embodiments according to the present invention, the symptoms alleviated include pain, inflammation, itching, numbness, nausea, vomiting, vertigo, depression, anxiety, and psychosis, or a combination of two or more of these symptoms. In embodiments according to the present invention, the disease state to be treated is a proliferative disease, such as psoriasis, or a neoplastic disease, such as melanoma, Hodgkins disease, non-Hodgkins lymphoma, or cancer. In such embodiments, the method according to the present invention causes a reduction in symptoms, such as pain, and/or slows or ceases progress of the disease by slowing or halting cell division of the disease cells, and/or induces remission of the disease by selectively killing disease cells or by slowing disease cell proliferation sufficiently to allow the patient's immune system to combat the disease.

The method of treatment according to the present invention may be used to treat various symptoms and disease states, such as pain, inflammation, and itching, either by themselves or concomitant with an underlying disease condition. Other disease states that may be treated by a method according to the present invention include proliferative diseases, such as psoriasis, neoplastic diseases, such as melanoma, lymphomas, sarcomas, and carcinomas, psychotic disorders, including schizophrenia, bipolar disorder, anxiety, depression, euphoria, psychosis, phobias, eating disorders, and substance addiction, gastrointestinal disorders such as irritable bowel syndrome, nausea, vomiting, Crohn's disease, etc.

Also included in ailments which may be treated by the subject method are disorders specific to non-humans, such as mange.

V. Exemplary Formulations and Preparations

Formulations of the present invention are suitable for topical (including buccal and sublingual) administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the codrug which produces a therapeutic effect. Generally, out of one hundred percent, this amount will range from about 1 percent to about ninety-nine percent of active ingredient, preferably from about 5 percent to about 70 percent, most preferably from about 10 per cent to about 30 percent.

Methods of preparing these formulations or compositions include the step of bringing into association a codrug of the present invention with the carrier and, optionally, one or more accessory ingredients. In general, the formulations are prepared by uniformly and intimately bringing into association a codrug of the present invention with liquid carriers, or finely divided solid carriers, or both, and then, if necessary, shaping the product.

Suspensions, in addition to the codrugs, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In certain embodiments according to the present invention, the pharmaceutical compositions comprise codrugs, wherein said codrugs comprise a residue of an antiproliferative compound or salt thereof linked directly or indirectly to a residue of a corticosteroid antiinflammatory agent or salt thereof, in an amount convenient for therapeutic administration, optionally in admixture with one or more pharmaceutically acceptable adjuvants, excipients, diluents, carriers, or dispersants.The adjuvant, excipient, diluent, carrier, or dispersant will vary depending upon the condition to be treated, the structure of the codrug, the desired mode of delivery, etc. Exemplary adjuvants include the aforementioned polymers and oils, as well a liposomes dispersed in aqueous solutions.

Codrugs according to the present invention may be prepared in free form, or may be prepared as salts, such as mineral acid, carboxylic acid, ammonium hydroxide or amine salts thereof. Codrugs according to the present invention may be prepared as amorphous or crystalline forms, and may be in the form of anhydrates or hydrates. Codrugs according to the present invention may be present as prodrugs, such as esters. In each of these cases, the critical feature is that a codrug according to the present invention be stable under some conditions other than physiologic conditions, and be capable of decomposing under physiologic conditions to form first and second constituent moieties, which moieties may be the same or different, as discussed above.

The codrugs of the present invention may also be provided in the form of prodrugs, e.g., to protect a biologically active moiety from being altered while passing through a hostile environment, such as the stratum comeum. Prodrugs can be prepared by forming covalent linkages between the biologically active moiety and a modifier. See, for example, Balant at al., Eur. J. Drug Metab. Pharmacokinetics, 1990, 15(2), 143-153. The linkage is usually designed to be broken under defined circumstances, e.g., pH changes or exposure to specific enzymes. The covalent linkage of the biologically active moiety to a modifier essentially creates a new molecule with new properties such as an altered log P value and/or as well as a new spatial configuration. The new molecule can have different solubility properties and be less susceptible to enzymatic digestion. For general references on prodrug design and preparation, see: Bundraard, Design of Prodrugs, Elsevier Science Pub. Co., N.Y. (1985), and Prodrugs as Novel Drug Delivery Systems Symposium, 168^{th} Annual Meeting, American Chemical Society, Atlantic City, N.J., Eds. T. Higuchi and V. Stella, ACS Symposium Series 14, 1975, which are herein incorporated by reference.

Prodrugs of amine-containing moieties are well known in the art and have been prepared, e.g., by reacting the amine moiety of a moiety with a carboxylic acid, acid chloride, chloroformate, or sulfonyl chloride modifiers, and the like, resulting in the formation of amides, sulfonamides, carboxyamides, carbamates, and similar compounds. See, for example, Abuchowski et al., J. Biol. Chem. 1977, 252, 3578-358; Senter et al., J. Org. Chem., 1990, 55, 2975-2978; Amsberry et al., J. Org. Chem., 1990, 55, 5867-5877; Klotz, Clin. Pharmacokinetics, 1985, 10, 285-302, which are herein incorporated by reference. Similar and other protocols may be followed for the formation of prodrugs of the codrugs of the present invention.

Polymers useful in a composition according to the present invention include biologically tolerated polymers that are permeable to a codrug according to the present invention, or that is permeable to the codrug and cleavage products thereof after the codrug has been cleaved, or that is bioerodible so that it releases the codrug according to the present invention in a sustained-release manner. In preferred embodiments according to the present invention, the polymer has a permeability such that it is not the principal rate-determining factor in the rate of release of the codrug according to the present invention from the polymer. In some embodiments, the polymer may have an effect on release rate or stability. In some embodiments according to the present invention, the polymer is non-bioerodible. Examples of non-bioerodible polymers useful in the present invention include polyvinylalcohol and polyurethane. In other embodiments of the present invention, the polymer is bioerodible. Examples of bioerodible polymers useful in the present invention include polyanhydride, polylactic acid, polyglycolic acid, polyorthoester, polyalkylcyanoacrylate or derivatives and copolymers thereof. The skilled artisan will recognize that the choice of bioerodibility or non-bioerodibility of the polymer depends upon the final physical form of the system, as described in greater detail below. Other exemplary polymers include polysilicone and polymers derived from hyaluronic acid. The skilled artisan will understand that the polymer according to the present invention is prepared under conditions suitable to impart permeability such that it is not the principal rate determining factor in the release of the low solubility agent from the polymer.

Moreover, suitable polymers include naturally occurring (collagen, hyaluronic acid) or synthetic materials that are biologically compatible with bodily fluids and mammalian tissues, and essentially insoluble in bodily fluids with which the polymer will come in contact. In addition, the suitable polymers may also essentially prevent interaction between the low solubility agent dispersed/suspended in the polymer and proteinaceous components in the bodily fluid.

Other suitable polymers include polypropylene, polyester, polyethylene vinyl acetate (PVA), polyethylene oxide (PEO), polypropylene oxide, polycarboxylic acids, polyalkylacrylates, cellulose ethers, polyalkyl-alkylacrylate copolymers, polyester-polyurethane block copolymers, polyether-polyurethane block copolymers, polydioxanone, poly—(β-hydroxybutyrate), polylactic acid (PLA), polycaprolactone, polyglycolic acid, and PEO-PLA copolymers.

Further suitable polymers are set forth in U.S. Pat. No. 6,051,576, issued on Apr. 18, 2000, to Ashton et al., which is expressly incorporated herein by reference.

Typical compositions according to the present invention include a wide variety of physical forms. These include, but are not limited to, solutions, lotions, creams, oils, gels, sticks, sprays, ointments, balms, shampoo, and pastes. Generally, such carrier systems can be described as being solutions, emulsions, gels, solids, and aerosols. The compositions may be applied topically to the skin, or may be applied in the form of a transdermal delivery device, such as a microneedle, a patch, bandage, or gauze pad known in the art.

The ointments, pastes, creams and gels may contain, in addition to a codrug composition of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a codrug composition of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Solvents are generally employed in the preparation of suitable topical compositions according to the present invention. Such solvents can either be aqueous or organic based, although pharmaceutically acceptable hydrophobic solvents are preferred. The solvent must be capable of having dispersed or dissolved therein the above-described active components while not being irritating to the animal being treated. Water forms the basis for all aqueous solvents, while suitable organic solvents include propylene glycol, butylene glycol, polyethylene glycol, polypropylene glycol, glycerol, 1,2,4-butanetriol, sorbitol esters, 1,2,6-hexanetriol, ethanol, isopropanol, butanediol, and mixtures thereof. Solvents can be included in the overall composition in amounts ranging from 0.1% to 99% and preferably from 2.0% to 75%. In some embodiments of the present invention, the compositions of the present invention are produced in the form of an emollient-containing composition. A wide variety of suitable emollients are known and may be used herein. In this regard, reference is made to U.S. Pat. No. 5,296,500, the disclosure of which is incorporated herein by reference.

In some embodiments of the present invention, the compositions are saturated or super-saturated in the codrug according to the present invention. In such cases, the codrug is partially solubilized in a carrier or vehicle, and is partially dispersed in the carrier or vehicle. In such embodiments, the composition according to the present invention comprises two phases, a continuous solvent-solute phase and a dispersed solid phase, wherein the solute and the dispersed solid both consist of the codrug. While not wishing to be bound by theory, it is believed that the solvent in such a system aids in delivery of the codrug to the skin, where the codrug readily penetrates the epidermis, thereby delivering the codrug to, or through, the skin. It is further believed that the dispersed solid phase is gradually taken up into the solvent as the solute passes across the dermal boundary. In such cases, residence time on the skin is enhanced for the codrug, and sustained release of the constituent moieties is accomplished.

In other embodiments, the compositions contain codrug fully dissolved in the solvent or carrier, forming a single, continuous, solution phase.

In some embodiments of the present invention, the compositions are formulated as lotions containing from about 0.01% to 10% of the codrugs described above. In other embodiments of the present invention, the compositions are formulated in a solution carrier system as a cream. A cream composition according to the present invention would preferably comprise from about 0.1% to 15% and preferably from 1% to 5% of the above described active ingredients. Lotions and creams can be formulated as emulsions as well as solutions.

In particular embodiments of the present invention, the codrugs described above are prepared as lotions or cream emulsions of the oil-in-water type or as a water-in-oil type. Suitable components of multi-phase emulsions of the water-in-oil type are disclosed in U.S. Pat. No. 4,254,105, the disclosure of which is incorporated herein by reference. The compositions may also be administered in liquid form, including in the form of liposomes suspended in liquid, as in the different type of sprays available in this industry.

In other embodiments according to the present invention, the codrugs set forth above are formulated as ointments. Suitable ointments may comprise simple bases of animal or vegetable oils, or semi-solid hydrocarbons (oleaginous). Suitable ointments may also comprise absorption ointment bases which absorb water to form emulsions. Ointment carriers may also be water soluble. An ointment may comprise from 1% to 99% of an emollient plus to about 0.1% to 99% of a thickening agent. See U.S. Pat. No. 5,296,500 and the citations contained therein for a more complete disclosure of the various ointment, cream and lotion formulations that may be used in combination with the above-disclosed codrugs.

The proportion of codrug in the compositions can vary from between about 0.01 wt. % to about 100 wt. %, more preferably from about 0.1 wt. % to about 99.9 wt. %, and especially from about 1.0 wt. % to about 99.0 wt. %.

In some embodiments according to the present invention, the compositions are applied to an area of the skin directly. In other embodiments according to the present invention, the compositions are applied to the skin via a transdermal delivery device, such as a microneedle, a patch, bandage, or gauze pad.

In some embodiments according to the present invention, the compositions are applied via a transdermal delivery patch. Suitable transdermal delivery patches comprise an impermeable backing layer, and a permeable layer for contacting the skin, the two layers being arranged so that between the two layers there is formed a reservoir for receiving the compositions according to the present invention. In some embodiments according to the present invention, the reservoir includes a carrier or vehicle.

Transdermal patches have the added advantage of providing controlled delivery of a codrug of the present invention to the body. Such dosage forms can be made by dissolving or dispersing the composition in the proper medium. Absorption enhancers can also be used to increase the flux of the composition across the skin. The rate of such flux can be controlled by either providing a rate-controlling membrane or dispersing the codrug in a polymer matrix or gel.

Suitable patch devices for use in the present invention may be bandages that can be attached to the surface of intact skin for prolonged periods of time to allow a desired systemic delivery of the codrugs. These transdermal patch devices occlude the skin and trap the biologically active moiety, together with volatiles and vehicle excipients, between the skin and an outer impermeable backing layer. The backing layer prevents evaporation or diffusion of vehicle excipients, volatiles, and biologically active moiety into an environment other than the target skin site.

“Carriers” or “vehicles” preferably refer to carrier materials suitable for transdermal drug administration and include any such materials known in the art, such as any liquid, gel solvent, liquid diluent, solubilizer, or the like, which is non-toxic, and which does not interact with other components of the composition in a deleterious manner. Examples of suitable carriers for use herein include water, silicone, liquid sugars, waxes, oils, petroleum jelly, and a variety of other materials. The term “carrier” or “vehicle” can also refer to crystallization inhibitors, or other types of additives useful for facilitating transdermal drug delivery.

In some embodiments according to the present invention, the carrier or vehicle includes one or more solvents, such as C₂-C₁₀ alcohols, such as hexanol, cyclohexanol, benzyl alcohol, 1,2-butanediol, glycerol, and amyl alcohol; C₅-C₁₀ hydrocarbons such as n-hexane, cyclohexane, and ethylbenzene; C₄-C₁₀ aldehydes and ketones, such as heptylaldehyde, cyclohexanone, and benzylaldehyde; C₄-C₁₀ esters, such as amyl acetate and benzyl propionate; ethereal oils, such as oil of eucalyptus, oil of rue, cumin oil, limonene, thymol, and 1-pinene; halogenated hydrocarbons having 2-8 carbon atoms, such as 1-chlorohexane, 1-bromohexane, and chlorocyclohexane. Suitable solvents are set forth in U.S. Pat. No. 3,598,122, which is expressly incorporated herein by reference.

Examples of oils comprise fats and oils such as olive oil and hydrogenated oils; waxes such as beeswax and lanolin; hydrocarbons such as liquid paraffin, ceresin, and squalane; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearate, sodium cetyl sulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g., the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants comprise butylated hydroxytoluene, butylated hydroxyanisole, propyl gallate, citric acid and ethoxyquin; examples of chelating agents include disodium edetate and ethanehydroxy diphosphate; examples of buffers comprise citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. The reservoir may be a void, or may include one or more layers of a suitable material for physically stabilizing the compositions according to the present invention. Suitable materials for the reservoir layer include, for example, polysiloxanes, polyisobutylenes, polyurethanes, plasticized ethylenevinyl acetate copolymers, low molecular weight polyether amide block polymers (e.g., PEBAX), tacky rubbers, such as polyisobutene, polystyrene-isoprene copolymers, polystyrene-butadiene copolymers, and mixtures thereof. The reservoir layer may comprise adhesive materials such as polyisobutylenes, silicones, polyurethanes, and polyacrylates, with polyisobutylenes particularly preferred.

In some embodiments according to the present invention, the compositions are applied via a transdermal delivery microneedle device. The microneedles of the device can be constructed from a variety of materials, including metals, ceramics, semiconductors, organics, polymers, and composites. Preferred materials of construction include pharmaceutical grade stainless steel, gold, titanium, nickel, iron, gold, tin, chromium, copper, alloys of these or other metals, silicon, silicon dioxide, and polymers. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polymethacrylic acid, ethylenevinyl acetate, polytetrafluorethylene and polyesters.

Generally, the microneedles should have the mechanical strength to remain intact for delivery of biologically active moieties, and to serve as a conduit for the collection of biological fluid and/or tissue, while being inserted into the skin, while remaining in place for up to a number of days, and while being removed. In certain embodiments, the microneedles maybe formed of biodegradable polymers. However, for these embodiments that employ biodegratable materials, the mechanical requirement may be less stringent.

The microneedles can be formed of a porous solid, with or without a sealed coating or exterior portion, or hollow. As used herein, the term “porous” means having pores or voids throughout at least a portion of the microneedle structure, sufficiently large and sufficiently interconnected to permit passage of fluid and/or solid materials through the microneedle. As used herein, the term “hollow” means having one or more substantially annular bores or channels through the interior of the microneedle structure, having a diameter sufficiently large to permit passage of fluid and/or solid materials through the microneedle. The annular bores may extend throughout all or a portion of the needle in the direction of the tip to the base, extending parallel to the direction of the needle or branching or exiting at a side of the needle, as appropriate. A solid or porous microneedle can be hollow. One of skill in the art can select the appropriate porosity and/or bore features required for specific applications. For example, one can adjust the pore size or bore diameter to permit passage of the particular material to be transported through the microneedle device.

The microneedles can have straight or tapered shafts. A hollow microneedle that has a substantially uniform diameter, which needle does not taper to a point, is referred to herein as a “microtube.” As used herein, the term “microneedle” includes, although is not limited to both microtubes and tapered needles unless otherwise indicated. In a preferred embodiment, the diameter of the microneedle is greatest at the base end of the microneedle and tapers to a point at the end distal the base. The microneedle can also be fabricated to have a shaft that includes both a straight (untapered) portion and a tapered portion.

The microneedles can be formed with shafts that have a circular cross-section in the perpendicular, or the cross-section can be non-circular. For example, the cross-section of the microneedle can be polygonal (e.g., star-shaped, square, triangular), oblong, or another shape. The shaft can have one or more bores. The cross-sectional dimensions typically are between about 10 nm and 1 mm, preferably between 1 micron and 200 microns, and more preferably between 10 and 100 μm. The outer diameter is typically between about 10 μm and about 100 μm, and the inner diameter is typically between about 3 μm and about 80 μm.

The length of the microneedles typically is between about 1 μm and 1 mm, preferably between 10 microns and 500 microns, and more preferably between 30 μm and 200 μm. The length is selected for the particular application, accounting for both an inserted and uninserted portion. An array of microneedles can include a mixture of microneedles having, for example, various lengths, outer diameters, inner diameters, cross-sectional shapes, and spacings between the microneedles.

In some embodiments according to the present invention, the codrugs according to the present invention are encapsulated in a hydrophobic polymer such as polyvinylchloride, optionally plasticized with one or more long-chain fatty acid amides, etc., plasticized nylon, non-plasticized soft nylon, silicone rubber, polyethylene, polyethylene terephthalate; or in a hydrophilic polymer, such as one or more esters of acrylic acid, methacrylic acid, modified collagen, cross-linked hydrophilic polyether gels, cross-linked polyvinylacetate, and cross-linked, partially hydrolyzed polyvinylacetate. Suitable encapsulating agents are set forth in U.S. Pat. No. 3,731,683, which is expressly incorporated herein by reference.

In certain embodiments of the invention, the carrier is composed of the foregoing materials to achieve a controlled occlusion of the skin, thereby resulting in optimal enhancement of biologically active moiety penetration across the skin with minimal skin irritation. In certain embodiments, the reservoir matrix may include a dispersing agent that aids in maintaining a particulate phase comprising the codrugs dispersed in the continuous phase. In other embodiments, non-ionic excipients, such as lauric alcohol, propylene glycol monolaurate, myristyl lactate, lauryl lactate, or the like, facilitate dispersion.

The rate of biologically active moiety delivery across a dermal surface can be increased by transdermal delivery enhancers. Suitable transdermal delivery enhancers include proton-accepting solvents such as dimethylsulfoxide and dimethylacetamide. Other suitable transdermal delivery enhancers include 2-pyrrolidine, N,N-diethyl-m-toluamide (Deet), 1-dodecylazacycloheptan-2-one (Azone™), N,N-dimethylformamide, N-methyl-2-pyrrolidine, terpenes, surfactants, and calcium thioglycolate. However, difficulties remain with such dermal enhancers because the problem of irritation at the site of application has not been overcome.

Suitable dermal penetration enhancers include 1-5 carbon fatty acid esters of para-aminobenzoic acid, isopropyl palmitate, isopropyl myristate, ethanol, isobutyl alcohol, isobutyl alcohol, stearyl alcohol, glycerol, 2-pyrrolidone, urea, propylene glycol, oleic acid, palmitic acid, dimethyl sulfoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, 2-pyrrolidone-5-carboxylic acid, N,N-dimethyl-m-toluamide, urea, ethyl acetate, 1-dodecylazacycloheptan-2-one (Azone™), oleic acid, imidazoline, butylurea, and pyrrolidone carboxylic acid esters.

In some embodiments according to the present invention, the reservoir also includes a hydrogel. Suitable hydrogels for use in a patch according to the present invention include those well known in the art, such as soluble cellulose ethers, e.g., methylcellulose and cellulose derivatives. Other suitable hydrogel materials include blends of either N-vinyl lactam or a copolymer of N-vinyl lactam, an aqueous mixture of a radiation crosslinkable water-soluble polymer such as a polymer of N-vinyl-2-pyrrolidone and ethylene oxide, and a humectant, such as propylene glycol which may be used in a transdermal drug delivery system.

Suitable hydrogels may contain preservatives such as propyl paraben and methyl paraben.

Suitable materials for the permeable skin contact layer include microporous rate-controlling materials such as polyvinylchlorides, polyamides, methacrylic copolymers, polysulfones, halogenated polymers, polychloroethers, acetal polymers, acrylic resins, polyurethanes, polyimides, polybenzimidazoles, polyvinylacetate, aromatic, and aliphatic polyethers, cellulose esters, cellulose triacetate, cellulose, cellulose nitrate, epoxy resins, and olefins, such as polyethylenes and polypropylenes.

Dosage forms for the topical or transdermal administration of a codrug of this invention include powders, sprays, ointments, pastes, creams, lotions, gels, solutions, patches and inhalants. The codrug may be mixed under sterile conditions with a pharmaceutically acceptable carrier, and with any preservatives, buffers, or propellants which may be required.

Ophthalmic formulations, eye ointments, powders, solutions, and the like, are also contemplated as being within the scope of this invention.

As set out above, certain embodiments of the present codrugs may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of codrugs of the present invention. These salts can be prepared in situ during the final isolation and purification of the codrugs of the invention, or by separately reacting a purified codrug of the invention in its free base form with a suitable organic or inorganic acid, and isolating the salt thus formed. Representative salts include the hydrobromide, hydrochloride, sulfate, bisulfate, formate, borate, phosphate, nitrate, acetate, valerate, oleate, palmitate, stearate, laurate, benzoate, lactate, phosphonate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject codrugs include the conventional nontoxic salts or quaternary ammonium salts of the codrugs, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the codrugs of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of codrugs of the present invention. These salts can likewise be prepared in situ during the final isolation and purification of the codrugs, or by separately reacting the purified codrug in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers, surfactants, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

As appropriate compositions for topical application there may be cited all compositions usually employed for topically administering therapeutics, e.g., creams, jellies, dressings, shampoos, tinctures, pastes, ointments, salves, powders, liquid or semiliquid formulations, and the like. Application of said compositions may be by aerosol, e.g., with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, pastes, jellies, ointments, and the like will conveniently be used.

It is especially advantageous to formulate the subject compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are powders packets, patches, suspensions, and the like, and segregated multiples thereof.

For preparing ointments, creams, toilet waters, skin milks, and the like, typically from 0.01 to 10% in particular from 0.1 to 5% and more in particular from 0.2 to 2.5% of the active ingredient, e.g., of a codrug, will be incorporated in the compositions. In ointments or creams, the carrier for example consists of 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% or in particular 0.5 to 1% of a surfactant, 1 to 20%, in particular 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume. In the aforementioned preparations, all % symbols refer to weight by weight percentage.

Particular compositions for use in the methods of the present invention are those wherein a codrug is formulated in liposome-containing compositions. Liposomes are artificial vesicles formed by amphiphatic molecules such as polar lipids, for example, phosphatidyl cholines, ethanolamines and serines, sphingomyelins, cardiolipins, plasmalogens, phosphatidic acids and cerebiosides. Liposomes are formed when suitable amphiphathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

The single bilayered liposomes containing the encapsulated codrug can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methylcellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. The preferred metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose.

The organic component consists of a suitable non-toxic, pharmaceutically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatydylserine, phosphatidylethanol-amine, phosphatidylinositol, lysophosphatidylcholine and phospha-tidyl glycerol, for example. Other lipophilic additives may be employed in order to selectively modify the characteristics of the liposomes. Examples of such other additives include stearylamine, phosphatidic acid, tocopherol, cholesterol, and lanolin extracts.

In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Apart from the above-described compositions, use may be made of covers, e.g. plasters, bandages, dressings, gauze pads and the like, containing an appropriate amount of a codrug. In some cases use may be made of plasters, bandages, dressings, gauze pads and the like which have been impregnated with a topical formulation containing the therapeutic formulation.

Equivalents

Those skilled in the art will recognize, or be able to ascertain, using no more than routine experimentation, numerous equivalents to the specific biologically active moieties, methods, diluents, polymers, and salts described herein. Such equivalents are considered to be within the scope of this invention.

We claim:

1. A pharmaceutical composition comprising a codrug, or a pharmaceutically acceptable salt or prodrug thereof, for topical administration of at least one biologically active moiety, which codrug comprises:
a) at least two constituent moieties, each moiety being a residue of a biologically active compound or a prodrug thereof, including a first constituent moiety and a second constituent moiety;
b) a linkage covalently linking said at least two constituent moieties to form said codrug, wherein said linkage is cleaved under physiological conditions after the codrug has been transported into or across the epidermal layer to regenerate said constituent moieties;
wherein the pH of the composition is less than about 7, and the codrug exhibits improved dermal uptake relative to at least one of the constituent moieties.

2. A pharmaceutical composition comprising a codrug, a pharmaceutically acceptable salt, or prodrug thereof, for topical administration of at least one biologically active moiety, which codrug comprises:
a) at least two constituent moieties, each moiety being a residue of a biologically active compound or a prodrug thereof, including a first constituent moiety and a second constituent moiety;
b) a linkage covalently linking said at least two constituent moieties to form said codrug, wherein said linkage is cleaved under physiological conditions after the codrug has been transported into or across the epidermal layer to regenerate said constituent moieties;
wherein the codrug has a log P value from about 1 to about 8, and the codrug exhibits improved dermal uptake relative to at least one of the constituent moieties

3. The pharmaceutical composition according to claim 1 or 2, wherein the first constituent moiety is selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds.

4. The pharmaceutical composition according to claim 3, wherein the second constituent moiety is selected from antidepressant compounds, analgesic compounds, antiinflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds.

5. The pharmaceutical composition according to claim 1 or 2, wherein the codrug has the following structural formula:
R₁−L−(R₂)ₙ
wherein the first constituent moiety is R₁;
the second constituent moiety is R₂;
R₁ and R₂ each represent, independently, a residue of a compound selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds;
n is an integer of from 1 to 4;
and L is selected from a direct bond and a linking group;
wherein the codrug has a log P value from about 1 to about 8, and the codrug exhibits improved dermal uptake relative to at least one of the constituent moieties.

6. The pharmaceutical composition according to claim 1 or 2, wherein the codrug has the following structural formula:
R₁−(L−R₂)ₙ
wherein the first constituent moiety is R₁;
the second constituent moiety is R₂;
R₁ and R₂ each represent, independently, a residue of a compound selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds;
n is an integer of from 1 to 4;
and L is selected from a direct bond and a linking group;
wherein the codrug has a log P value from about 1 to about 8, and the codrug exhibits improved dermal uptake relative to at least one of the constituent moieties.

7. The pharmaceutical composition according to claim 1 or 2, wherein the codrug has the following structural formula:
(R₁−L)ₘR₂(L₂−R₃)ₙ
wherein the first constituent moiety is R₁;
the second constituent moiety is R₂;
R₁, R₂, and R₃ each represent, independently, a residue of a compound selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds;
m is an integer of from 1 to 4; and
L and L₂ are each independently selected a direct bond and a linking group; wherein the codrug has a log P value from about 1 to about 8, and the codrug exhibits improved dermal uptake relative to at least one of the constituent moieties.

8. The pharmaceutical composition according to claim 5, 6, or 7, wherein R₂ is a residue of diclofenac, etodolac, ketorolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or salts thereof.

9. The pharmaceutical composition according to claim 5, 6, or 7, wherein R₁ is a residue of alitretinoin (9-cis-retinoic acid); amifostine; bexarotene (4-[1-(5,6,7,8-tetrahydro-3,5,5,8,8-pentamethyl-2-naphthalenyl) ethenyl] benzoic acid); bleomycin; capecitabine (5′-deoxy-5-fluoro-cytidine); chlorambucil; bleomycin; BCNU; cladribine; cytarabine; daunorubicin; docetaxel; doxorubicin; epirubicin; estramustine; etoposide; exemestane (6-methylenandrosta-1,4-diene-3,17-dione); fludarabine; 5-fluorouracil; gemcitabine; hydroxyurea; idarubicin; irinotecan; melphalan; methotrexate; mitoxantrone; paclitaxel; pentostatin; streptozocin; temozolamide; teniposide; tomudex; topotecan; valrubicin (N-trifluoroacetyladriamycin-14-valerate); or vinorelbine.

10. The pharmaceutical composition according to claim 5, 6, or 7, wherein R₂ is a residue of: 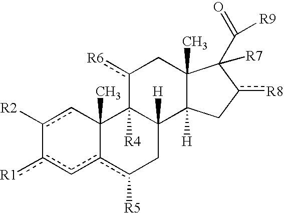
wherein R1 is ═O, —OH, or —(CH₂)₁₋₄Cl;
R2 is H, C₁₋₄alkyl, Cl, or Br;
R4 is H, F, or Cl;
R5 is H, F, Cl, CH₃, or —CHO;
R6 is H, OH, or Cl;
R7 is H, OH, CH₃, O—COCH₃, O(CO)OCH₂CH₃, O—(CO)-2-furanyl, or O—C(O)—(CH₂)₂CH₃;
R8 is H, CH₃, OH, ═CH₂, or together R7 and R8 form, together with the adjacent carbon atoms to which they are attached: 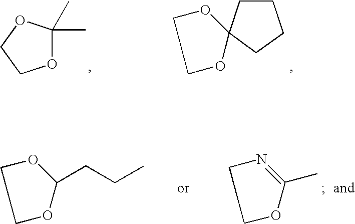
R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)-NH(CO)C₆H₅, or —S—CH₂—F; and
wherein the bonds indicated by [symbol]
are either double or single bonds.

11. The pharmaceutical composition according to claim 5, 6, or 7, wherein R₂ is a residue of 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts thereof.

12. The pharmaceutical composition according to claim 1 or 2, further comprising a carrier, an excipient, a solvent, an adjuvant, a diluent, a dispersant, or a surfactant.

13. The pharmaceutical composition according to claim 12, said carrier comprising a biocompatible polymer.

14. The pharmaceutical composition according to claim 13, wherein the polymer comprises PVA.

15. The pharmaceutical composition according to claim 1 or 2, wherein composition has a pH of less than about 6.

16. The pharmaceutical composition according to claim 1 or 2, wherein at least one of the constituent moieties has a log P value at least 1 log P unit less than the log P value of the codrug.

17. The pharmaceutical composition according to claim 1 or 2, wherein the codrug has a log P value from about 1 to about 3.

18. The pharmaceutical composition according to claim 1 or 2, wherein the codrug has a log P value from about 3 to about 6.

19. The pharmaceutical composition according to claim 1 or 2, wherein the composition is for local dermal delivery.

20. The pharmaceutical composition according to claim 1 or 2, wherein the composition is for systemic transdermal delivery.

21. The pharmaceutical composition according to claim 1 or 2, wherein the first constituent moiety is the same as the second constituent moiety.

22. The pharmaceutical composition according to claim 1 or 2, wherein the first constituent moiety is different from the second constituent moiety.

23. The pharmaceutical composition according to claim 1 or 2, wherein the first and second constituent moieties are directly linked through a covalent bond formed between a functional group of the first constituent moiety and a functional group of the second constituent moiety.

24. The pharmaceutical composition according to claim 1 or 2, wherein the first and second constituent moieties are linked to one another via a linking group that is covalently bonded to the first and second constituent moieties via functional groups thereon.

25. The pharmaceutical composition according to claim 3 or 4, wherein the first constituent moiety is a corticosteroid.

26. The pharmaceutical composition according to claim 3 or 4, wherein the second constituent moiety is a corticosteroid, a keratolytic compound, a skin-treating compound, an antiproliferative compound, or a non-steroidal anti-inflammatory compound.

27. The pharmaceutical composition according to claim 25 or 26, wherein the corticosteroid is selected from triamcinolone acetonide, fluocinolone acetate, fluocinolone acetonide, cortisone, hydrocortisone, and hydrocortisone ester.

28. The pharmaceutical composition according to claim 1 or 2, wherein the first constituent moiety is an antiproliferative agent and the second constituent moiety is a non-steroidal anti-inflammatory agent, with the proviso that the first constituent moiety is not floxuridine, and with the further proviso that when the first constituent moiety is 5-fluororuracil, the second constituent moiety is not flurbiprofen or indomethacin.

29. The pharmaceutical composition according to claim 1 or 2, wherein the first constituent moiety is an antiproliferative agent and the constituent moiety is a corticosteroid agent, with the proviso that when the antiproliferative agent is 5-fluorouracil, the corticosteroid is not fluocinolone acetonide, triamcinolone, triamcinolone acetonide, desoximetasone, or hydrocortisone-17-butyrate, and with the further proviso that the antiproliferative agent is not a 1-β-arabinofuranosylcytosine derivative.

30. A method of treatment, comprising administering to a patient in need thereof a therapeutically effective amount of a composition according to claim 1 or 2, or a pharmaceutically acceptable salt thereof.

31. The method according to claim 30 wherein the composition is for local dermal delivery.

32. The method according to claim 30, wherein the composition is for systemic transdermal delivery.

33. The method according to claim 30, wherein the first constituent moiety is the same as the second constituent moiety.

34. The method according to claim 30, wherein the first constituent moiety is different from the second constituent moiety.

35. The method according to claim 30, wherein the first and second constituent moieties are directly linked through a covalent bond formed between a functional group of the first constituent moiety and a functional group of the second constituent moiety.

36. The method according to claim 30, wherein the first and second constituent moieties are linked to one another via a linking group that is covalently bonded to the first and second constituent moieties via functional groups thereon.

37. The method according to claim 30, wherein the therapeutically effective amount is an amount effective to produce an analgesic, an anti-inflammatory, an antibiotic, an anti-fungal, an antiviral, an antiproliferative, a skin-treating, a sunscreen, a skin protecting, an antimetabolite, an antipsoriatic, and/or keratolytic effect in the patient.

38. A device for delivery of one or more biologically active moieties to or through the skin, the device comprising:
a composition according to claim 1 and a means for releasing the codrug into or across the skin.

39. The device according to claim 38, wherein the means for releasing the codrug into or across the skin is a microneedle, a bandage, a gauze pad, or a patch.

40. The device according to claim 39, wherein the means for releasing the codrug into or across the skin is a patch, said patch comprising an impermeable backing layer, a permeable skin contact layer, and a reservoir containing said composition.

41. The device according to claim 40, wherein the reservoir comprises one or more solvents, permeability enhancers, hydrogels, or non-hydrophilic polymers.
